SEQUENCE LISTING (48 sequences)

SEQ ID NO: 1 - protein, 240 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 2 - protein, 214 aa, Homo sapiens
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 4 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 5 - protein, 233 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCAA

SEQ ID NO: 6 - protein, 233 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCAA

SEQ ID NO: 7 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQCLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 8 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 9 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQCDDFATYYCQQYNSYPYTFGQGTKLEVKRTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGES

SEQ ID NO: 10 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 11 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 12 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 13 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 14 - protein, 235 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGCAA

SEQ ID NO: 15 - protein, 235 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVESKYGCAA

SEQ ID NO: 16 - protein, 231 aa, Homo sapiens
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 17 - protein, 215 aa, Homo sapiens
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 18 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKCLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCAA

SEQ ID NO: 19 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGCGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAESS

SEQ ID NO: 20 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 21 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 22 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKCLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 23 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGCGTKLTVLGQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 24 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVETCPAVLQSSGLYSLSSVVTVPSSSLGTQTYICNVNHKPSNTKVDKKVEPKSSDKTHTCAA

SEQ ID NO: 25 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGQPKAAPSVTLFPPSSEELQANKATLVCLVSDFYPGAVTVAWKADGSPVKVGVEDTKPSKQSNNKYAACSYLSLTPEQWKSHRSYSCRVTHEGSTVEKTVAPAESS

SEQ ID NO: 26 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVFTCPAVLQSSGLYSLSSVVTVPSSSLGTQTYICNVNHKPSNTKVDKKVEPKSSDKTHTCAA

SEQ ID NO: 27 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGQPKAAPSVTLFPPSSEELQANKATLVCLVSDFYPGAVTVAWKADGSPVKVGVELTKPSKQSNNKYVACSYLSLTPEQWKSHRSYSCRVTHEGSTVEKTVAPAESS

SEQ ID NO: 28 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKLLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 29 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGLGTKLTVLGQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 30 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKLLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVETCPAVLQSSGLYSLSSVVTVPSSSLGTQTYICNVNHKPSNTKVDKKVEPKSSDKTHTCAA

SEQ ID NO: 31 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGLGTKLTVLGQPKAAPSVTLFPPSSEELQANKATLVCLVSDFYPGAVTVAWKADGSPVKVGVEDTKPSKQSNNKYAACSYLSLTPEQWKSHRSYSCRVTHEGSTVEKTVAPAESS

SEQ ID NO: 32 - protein, 231 aa, Artificial Sequence
  source: /note=“Description of Artificial Sequence Synthetic polypeptide”
QVQLVQSGGGLVQPGGSLRLSCAASGFTFSSYAMHWVRQAPGKLLEWVTVISPDGHNTYYADSVKGRFTISRDNSKNTLYLQMNSLRAEDTAVYYCARASVGATGPFDIWGQGTLVTVSSASTKGPSVFPLAPSSKSTSGGTAALGCLVKDYFPEPVTVSWNSGALTSGVFTCPAVLQSSGLYSLSSVVTVPSSSLGTQTYICNVNHKPSNTKVDKKVEPKSSDKTHTCAA

SEQ ID NO: 33 - protein, 215 aa, Artificial Sequence
  source: /note=“Description of Artificial Sequence Synthetic polypeptide”
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGLGTKLTVLGQPKAAPSVTLFPPSSEELQANKATLVCLVSDFYPGAVTVAWKADGSPVKVGVELTKPSKQSNNKYVACSYLSLTPEQWKSHRSYSCRVTHEGSTVEKTVAPAESS

SEQ ID NO: 34 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVETCPAVLQSSGLYSLSSVVTVPSSSLGTQTYICNVNHKPSNTKVDKKVEPKSSDKTHTCAA

SEQ ID NO: 35 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQEDVTEQDSKDSTYSLCSTLTLSKADYEKHKVYACEVTHQGLSSPVTKSFNRGES

SEQ ID NO: 36 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVFTCPAVLQSSGLYSLSSVVTVPSSSLGTQTYICNVNHKPSNTKVDKKVEPKSSDKTHTCAA

SEQ ID NO: 37 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQELVTEQDSKDSTYVLCSTLTLSKADYEKHKVYACEVTHQGLSSPVTKSFNRGES

SEQ ID NO: 38 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQLLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 39 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQLTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 40 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQLLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVETCPAVLQSSGLYSLSSVVTVPSSSLGTQTYICNVNHKPSNTKVDKKVEPKSSDKTHTCAA

SEQ ID NO: 41 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQLTKLEIKRTVAAPSVFIFPPSDEQLKSGTASVVCLLNNFYPREAKVQWKVDNALQSGNSQEDVTEQDSKDSTYSLCSTLTLSKADYEKHKVYACEVTHQGLSSPVTKSFNRGES

SEQ ID NO: 42 - protein, 240 aa, Artificial Sequence
  source: /note=“Description of Artificial Sequence Synthetic polypeptide”
QVQLVQSGAEVKKPGSSVKVSCKASGGTFSSYAISWVRQAPGQLLEWMGGIIPIFGTANYAQKFQGRVTITADESTSTAYMELSSLRSEDTAVYYCARDPKRDDYIWGSYRPQYAFDIWGQGTMVTVSSASTKGPSVFPLAPSSKSTSGGTAALGCLVKDYFPEPVTVSWNSGALTSGVFTCPAVLQSSGLYSLSSVVTVPSSSLGTQTYICNVNHKPSNTKVDKKVEPKSSDKTHTCAA

SEQ ID NO: 43 - protein, 214 aa, Artificial Sequence
  source: /note=“Description of Artificial Sequence Synthetic polypeptide”
AIQLTQSPSTLSASVGDRVTITCRASQSISSWLAWYQQKPGKAPKLLIYKASSLESGVPSRFSGSGSGTEFTLTISSLQPDDFATYYCQQYNSYPYTFGQLTKLEIKRTVAAPSVFIFPPSDEQLKSGTASVVCLLNNFYPREAKVQWKVDNALQSGNSQELVTEQDSKDSTYVLCSTLTLSKADYEKHKVYACEVTHQGLSSPVTKSFNRGES

SEQ ID NO: 44 - protein, 8 aa, Artificial Sequence
  source: /note=“Description of Artificial Sequence Synthetic peptide”
DKTHTCAA

SEQ ID NO: 45 - protein, 6 aa, Artificial Sequence
  source: /note=“Description of Artificial Sequence Synthetic peptide”
ERKCAA

SEQ ID NO: 46 - protein, 8 aa, Artificial Sequence
  source: /note=“Description of Artificial Sequence Synthetic peptide”
ESKYGCAA

SEQ ID NO: 47 - protein, 516 aa, Artificial Sequence
  source: /note=“Description of Artificial Sequence Synthetic polypeptide”
QGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNHHHHHH

SEQ ID NO: 48 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2018/0271998 A1
(43) Pub. Date: Sep. 27, 2018

(54) DISULFIDE-STABILIZED FABS

(71) Applicant: Merrimack Pharmaceuticals, Inc., Cambridge, MA (US)

(72) Inventors: Daryl C. DRUMMOND, Lincoln, MA (US); Melissa GEDDIE, Arlington, MA (US); Dmitri B. KIRPOTIN, Revere, MA (US); Alexey Alexandrovich LUGOVSKOY, Belmont, MA (US)

(21) Appl. No.: 15/780,986

(22) PCT Filed: Dec. 5, 2016
(86) PCT No.: PCT/US2016/064943

§ 371 Date: Jun. 1, 2018

Related U.S. Application Data
(60) Provisional application No. 62/263,368, filed on Dec. 4, 2015.

(51) Int. Cl. A61K 47/69; C07K 16/28; C07K 16/30; C07K 16/32

(52) U.S. Cl. CPC A61K 47/6913; C07K 16/2863; C07K 16/30; C07K 2317/94; C07K 2317/55; C07K 2317/624; C07K 16/32

(57) ABSTRACT

Provided are antibody fragments (Fabs) wherein native disulfide bonds are absent and engineered disulfide bonds have been introduced. Some fragments comprise further additional beneficial mutations. The fragments exhibit immuno specific binding and desirable stability properties, e.g., the fragments can be efficiently conjugated to effectors at high temperatures (e.g., >60° or >70° C.) without denaturing.

### RELATED APPLICATIONS

This application claims the benefit of U.S. Provisional Patent Application Ser. No. 62/263,368 filed Dec. 4, 2015. The entire contents of the above-referenced patent application is incorporated herein by this reference.

### BACKGROUND

Antibodies are extremely useful molecules, both in organisms in which they are naturally produced and as laboratory reagents and pharmaceuticals. One particularly valuable property of antibodies is their ability to bind tightly, with exquisite specificity, to any particular biomolecule, as well as to inorganic antigen targets. To effectively exploit some of the useful properties of antibodies, methods of engineering antibodies, and of conjugation antibodies to other molecules and to substrates have been developed.

In conjugating antibody fragments, such as Fv, Fab, Fab′, F(ab′)² and other antibody fragments, site-specific conjugations to the amino acid cysteine is often exploited. Cysteine is used because cysteines are rare in the antibody fragments and are typically not located at antigen binding sites within antibodies, and because cysteine contains a reactive sulfhydryl group. Cysteines long have been engineered at locations within antibodies where they do not naturally occur (see, e.g., U.S. Pat. Nos. 5,219,996, 5,677,425, 7,122,636, 8,053,562, and 8,066,994, and WIPO patent publications WO198901974, WO2005003169, WO2005003170, WO2005003171, WO200603448, WO2007010231, WO20080380024, WO2010107109, WO2011061492, WO2011118739, WO2013096291, WO2013093809, and WO2014124316; and European Patent Nos. EP2465871, EP0348442, and EP968291).

Targeted nanoparticles, exemplified by antibody-fragment conjugated immunoliposomes, represent a promising therapeutic strategy for treating human diseases, e.g., cancers. In constructing these targeted nanoparticles antibody fragments are often used rather than full length immunoglobulin molecules.

Single chain Fv (“scFv”) antibody fragments have been utilized in multiple immunoliposome constructs (e.g., U.S. Pat. Nos. 7,244,826 and 8,138,315; US Patent Publication No. 20100009390). However scFvs typically lack sufficient thermal stability (as evidenced by lack of denaturation in physiologic buffers up to a minimum of 60° C., and preferably ≥70° C.) to allow for their use in commercially feasible manufacturing processes. One useful process for attaching a targeting antibody to a liposome comprises the separate steps of (1) conjugation of antibody to lipopolymer, (2) manufacturing of liposomes containing a therapeutic agent, and (3) an elevated temperature (generally >60° C. and, depending on liposomal membrane lipid composition, sometimes >70° C.) incubation step that facilitates insertion of the lipopolymer moiety of the antibody-lipopolymer conjugate into the outer leaflet of the liposome bilayer (see, e.g., U.S. Pat. No. 6,210,707). This insertion step generally must be carried out at a temperature of at least 60-65° C., and for some membrane phospholipid compositions, over 70° C. Since many scFvs will denature (often irreversibly) at such temperatures in media required for the insertion step, obtaining antibody fragments targeted to any desired antigen that are stable under these conditions is critical to the manufacture of immunoliposomal products.

Fabs are antibody fragments that are typically more thermally stable than scFvs. Unfortunately, procedures used to manufacture immune-nanoparticles such as immunoliposomes include antibody conjugation, e.g., to lipopolymer. Such conjugation is typically effected via reaction with antibody cysteine residues, which requires reduction of a free cysteine in the antibody. This creates problems because antibody internal disulfides will also be reduced, often resulting in denaturation. Subsequent conjugation to such over-reduced antibodies yields heterogeneous products (often with reduced or abrogated antigen binding properties). In addition to conjugation to cysteines of reduced disulfides (which destroys secondary structure essential to antibody function) such conjugation products also comprising lower molecular weight impurities that are both difficult to characterize and may confer undesirable pharmacologic properties upon the conjugation product. Thus there is a need for improved Fabs that are suitable for conjugation, and for conjugates thereof. The following disclosure provides novel antibodies and antibody conjugates that address this need and provide additional benefits.

### SUMMARY

Disclosed herein are Fabs lacking at least one native disulfide bond that comprise at least one engineered disulfide bond located at one or more specific regions where disulfide bonds do not naturally occur within the Fab molecules. The engineered disulfide bonds stabilize the Fabs, e.g., during attachment of one or more effector moieties, and are positioned so as to facilitate effector attachment via an engineered cysteine residue within 10 amino acid residues from the carboxyl terminus (C-terminus) of the Fab heavy chain while minimizing effector attachment to any other Fab cysteine residue. Also disclosed are Fab conjugates comprising such engineered Fabs.

Particular embodiments include: A Fab comprising a heavy chain and a light chain and characterized in that there is not a cysteine at position 233 and at position 127 of the heavy chain and there is not a cysteine at position 214 of the light chain, and the heavy chain and the light chain are linked together by one or two heavy-chain-light-chain disulfide bonds, each of the one or two bonds connecting a different pair of engineered cysteines located at (i) position 44 of the heavy chain and position 100 of the light chain or (ii) position 174 of the heavy chain and position 176 of the light chain. Such Fabs may further comprise (i) glutamic acid at heavy chain position 172 and aspartic acid at light chain position 162 or (ii) phenylalanine at heavy chain position 172 and leucine at light chain position 162 or (iii) leucine at heavy chain position 44 and leucine at light chain position 100. Alternatively, such Fabs may further comprise leucine at heavy chain position 44 and leucine at light chain position 100, and i) glutamic acid at heavy chain position 172 and aspartic acid at light chain position 162 or (ii) phenylalanine at heavy chain position 172 and leucine at light chain position 162 and valine at light chain position 174. Exemplary Fabs comprise at least one cysteine within 10 amino acid residues of the C-terminus of the heavy chain. In various such Fabs this cysteine is comprised within an amino acid sequence of SEQ ID NO:44, SEQ ID NO:45, or SEQ ID NO:46, which sequence is located at (e.g., appended to) the C-terminus of the heavy chain. Each of the above disclosed Fabs may have a kappa light chain or a lambda light chain.

The thermostability of some of the disclosed Fabs, e.g., as measured by a thermal shift assay using a differential scanning fluorimetry readout, is comparable to a matched Fab in which there is not a cysteine at any of position 44 of the heavy chain, position 100 of the light chain, position 174 of the heavy chain and position 176 of the light chain, and which comprises a cysteine at position 233 or at position 127 of the heavy chain and a cysteine at position 214 of the light chain. Some of the disclosed Fabs have binding strength for target antigen that is at least (i.e., no less than) 75% or 85% of that of a matched Fab in which there is not a cysteine at any of position 44 of the heavy chain, position 100 of the light chain, position 174 of the heavy chain and position 176 of the light chain, and which comprises a cysteine at position 233 or at position 127 of the heavy chain and a cysteine at position 214 of the light chain.

Any of the above described Fabs may have a moiety (e.g., an effector) attached (conjugated) to at least one C-terminal cysteine. The moiety may be a lipid:drug complex or the liposome that may comprise a drug, e.g., a cytotoxin. The moiety may comprise a linker linking it to the cysteine, optionally a cleavable linker (e.g., a pH sensitive linker, a disulfide linker, an enzyme-sensitive linker) or a biodegradable linker. The linker may be a polyethylene glycol linker. The Fab beneficially exhibits no reduction, or no more than 5%, 10%, or 20% reduction in stability, e.g., as measured by a thermal shift assay using a differential scanning fluorimetry readout, during moiety conjugation, when compared to a matched native Fab. For example, the Fab exhibits a Tm of 65° C. or greater (e.g., a Tm of 70° C. or greater, 71° C. or greater, 72° C. or greater, 73° C. or greater, 74° C. or greater, 75° C. or greater, 76° C. or greater, 77° C. or greater, 78° C. or greater, 79° C. or greater, or 80° C. or greater) as measured by a thermal shift assay using a differential scanning fluorimetry readout.

Various exemplified Fabs include Fabs comprising: (a) a heavy chain having an amino acid sequence of SEQ ID NO:18 and a light chain having an amino acid sequence of SEQ ID NO:19, (b) a heavy chain having an amino acid sequence of SEQ ID NO:20 and a light chain having an amino acid sequence of SEQ ID NO:21, (c) a heavy chain having an amino acid sequence of SEQ ID NO:22 and a light chain having an amino acid sequence of SEQ ID NO:23, (d) a heavy chain having an amino acid sequence of SEQ ID NO:24 and a light chain having an amino acid sequence of SEQ ID NO:25, (e) a heavy chain having an amino acid sequence of SEQ ID NO:26 and a light chain having an amino acid sequence of SEQ ID NO:27, (f) a heavy chain having an amino acid sequence of SEQ ID NO:28 and a light chain having an amino acid sequence of SEQ ID NO:29, (g) a heavy chain having an amino acid sequence of SEQ ID NO:30 and a light chain having an amino acid sequence of SEQ ID NO:31, and (h) a heavy chain having an amino acid sequence of SEQ ID NO:32 and a light chain having an amino acid sequence of SEQ ID NO:33.

Also provided are pharmaceutical compositions comprising any of the above-disclosed Fabs together with one or more pharmaceutically acceptable excipients, diluents, or carriers.

Also provided are methods of preparing the above-described Fabs, in which methods attachment of the moiety is accomplished by a maleimide thiol reaction between a di-C₁₈ or distearoyl phosphoethanolamine-N-[maleimide] linker (e.g., a 2-distearoyl-sn-glycero-3-phosphoethanolamine-N-[maleimide(polyethylene glycol)] linker) and the cysteine. In some of the disclosed moiety-conjugated Fabs the moiety comprises a lipidic nanoparticle, e.g., a liposome, a lipid:nucleic acid complex, a lipid:drug complex, or a microemulsion droplet. The conjugation yield for the Fab is beneficially greater than 60% or 70%, and the number of free [SH]/Fab is beneficially less than 1.5 or less than 1.2.

In one aspect, a Fab is attached to a lipidic nanoparticle (e.g., a liposome, a lipid:nucleic acid complex, a lipid:drug complex, and a microemulsion droplet) by means of a linker molecule, the method comprising: attaching a Fab as described above to a linker molecule comprising a linear hydrophilic polymer chain having a first end and a second end, with, attached to the first end, a chemical group reacted with one or more functional groups on the Fab, and attached to the second end, a hydrophobic domain (optionally a lipid hydrophobic domain) and incubating the Fab-linker conjugate with the lipidic nanoparticle at a temperature of greater than 50, 60, or 70° C. for a time sufficient to permit the hydrophobic domain to become stably associated with the lipidic nanoparticle (e.g., by insertion into a lipid membrane comprised by the nanoparticle). The insertion efficiency of the conjugate into the lipid membrane is preferably greater than 80%, and more preferably greater than 90%. Insertion efficiency may be tested using approximately 100 nm diameter liposomes comprising cholesterol and 1,2-distearoyl-sn-phosphatidylcholine (DSPC), e.g., prepared essentially as described in Example 2 of U.S. Pat. No. 8,147,867, and Example 4 disclosed herein.

### BRIEF DESCRIPTION OF THE DRAWINGS

FIG. 1 shows SDS-PAGE analysis of various Fabs subjected to non-reducing, non-denaturing conditions (FIG. 1A) and to reducing, denaturing conditions (FIG. 1B). For both FIGS. 1A and 1B: lane M contains molecular weight markers; Lane 1, Fab 1; lane 2, Fab 2; lane 3, Fab 3; lane 4, Fab 4; lane 5, Fab 5; lane 6, Fab 6; lane 7, Fab 7; lane 8, Fab8; lane 9, Fab 9; lane 10, Fab 10.

FIG. 2A shows SDS-PAGE analysis of Fab constructs Fab 11, Fab 12, Fab 13, and Fab 14 subjected to non-reducing, non-denaturing conditions (lanes 1-4) and to reducing, denaturing conditions (lanes 6-9). Lane M contains molecular weight markers; lanes 1 and 6, Fab 11; lanes 2 and 7, Fab 12; lanes 3 and 8, Fab 13; lanes 4 and 9, Fab 14; no sample was loaded in lane 5. FIGS. 2B-2D show schematics of Fab constructs, illustrating the location of the disulfide bonds, such as in a wild-type Fab (Fab 11; FIG. 2B) and three engineered constructs having relocated disulfide bonds (Fab 12; FIG. 2C; Fab 13; FIG. 2D; Fab 14, FIG. 2E).

FIG. 3 shows SDS-PAGE analysis of Fabs subjected to non-reducing, non-denaturing conditions (FIG. 3A) and to reducing, denaturing conditions (FIG. 3B). For both FIGS. 3A and 3B: lane M contains molecular weight markers; Lane 1, Fab 11; lane 2, Fab 15; lane 3, Fab 16; lane 4, Fab 17; lane 5, Fab 18; lane 6, Fab 19.

FIG. 4 shows SDS-PAGE analysis of Fab constructs Fab 20, Fab 21, and Fab 22 subjected to non-reducing, non-denaturing conditions (lanes 1-3) and to reducing, denaturing conditions (lanes 5-7). Lane M contains molecular weight markers; lanes 1 and 5, Fab 20; lanes 2 and 6, Fab 21; lanes 3 and 7; Fab 22; no sample was loaded in lane 4.

FIG. 5 shows Ultrogel AcA34 chromatography elution profiles for mal-DSPE PEG-conjugated Fab 11, Fab 12, Fab 13, and Fab 14 constructs.

FIG. 6 shows SDS-PAGE analysis of conjugated and unconjugated Fab 11, Fab 12, Fab 13, and Fab 14 constructs under various conditions (as set forth in Table 1).

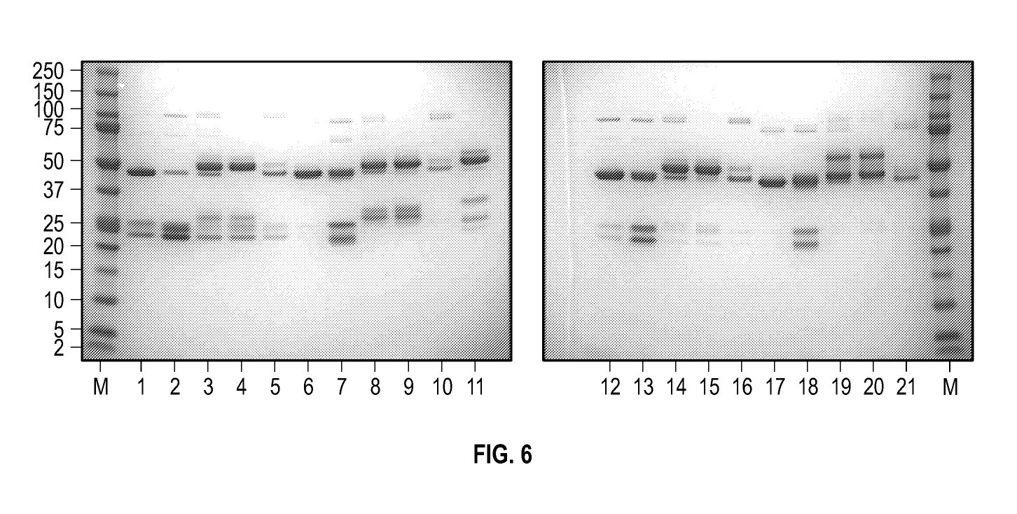

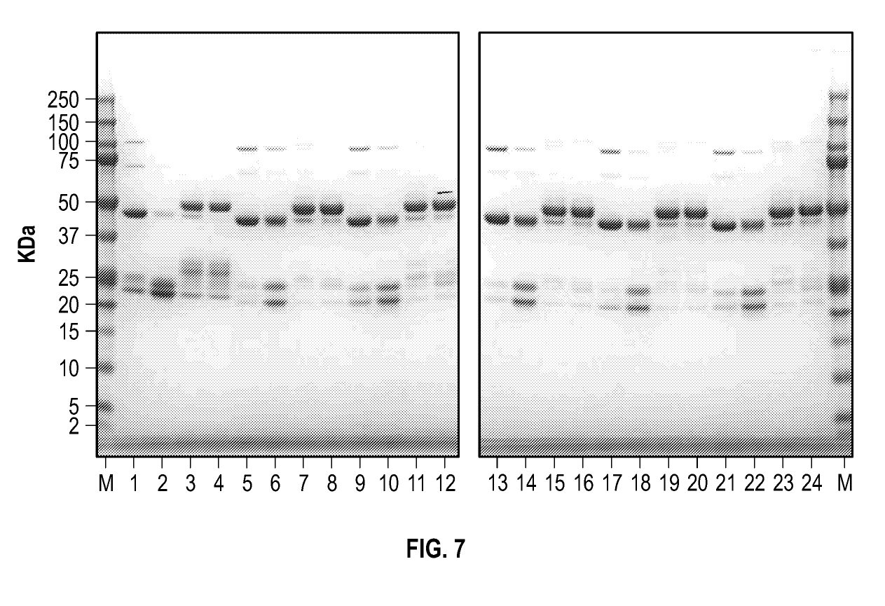

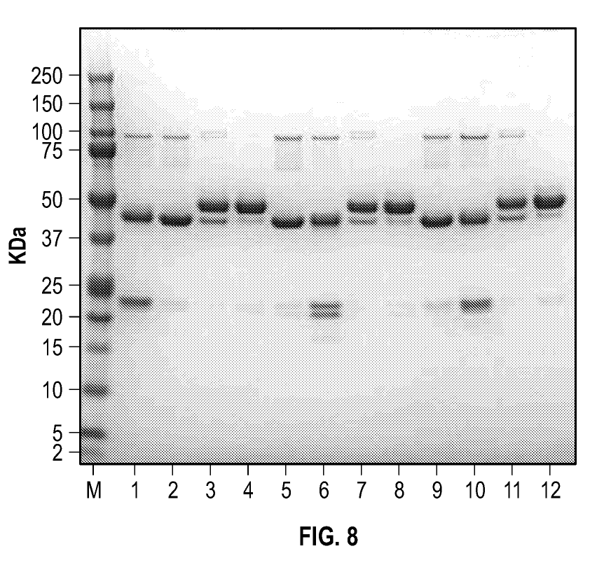

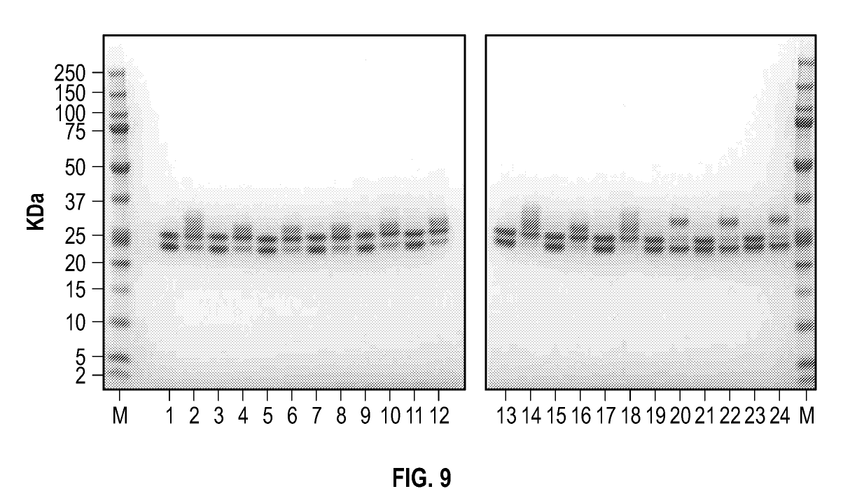

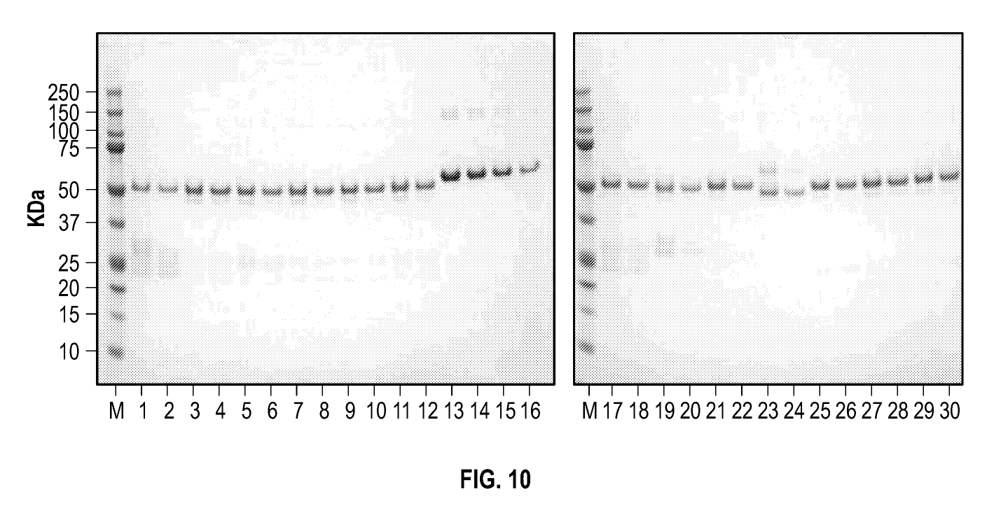

TABLE 1
Lane contents for FIG. 6

| Lane | Sample |
|---|---|
| M | Ladder |
| 1 | Fab 11 non-reduced |
| 2 | Fab 11 reduced |
| 3 | Fab 11 conjugation mix |
| 4 | Fab 11 purified conjugate |
| 5 | Fab 11 unconjugated fraction |
| 6 | Fab 12 non-reduced |
| 7 | Fab 12 reduced |
| 8 | Fab 12 conjugation mix |
| 9 | Fab 12 purified conjugate |
| 10 | Fab 12 unconjugated fraction |
| 11 | E1-PEG-DSPE purified conjugate (reference) |
| 12 | Fab 13 non-reduced |
| 13 | Fab 13 reduced |
| 14 | Fab 13 conjugation mix |
| 15 | Fab 13 purified conjugate |
| 16 | Fab 13 unconjugated fraction |
| 17 | Fab 14 non-reduced |
| 18 | Fab 14 reduced |
| 19 | Fab 14 conjugation mix |
| 20 | Fab 14 purified conjugate |
| 21 | Fab 14 unconjugated fraction |
| M | Ladder |

FIG. 7 shows SDS-PAGE analysis of the conjugated and unconjugated constructs Fab 11, Fab 15, Fab 16, Fab 17, Fab 18 and Fab 19 under various conditions (as set forth in Table 2).

TABLE 2
Lane contents for FIG. 7

| Lane | Sample |
|---|---|
| M | Ladder |
| 1 | Fab 11 non-reduced |
| 2 | Fab 11 reduced |
| 3 | Fab 11 conjugation mix |
| 4 | Fab 11 purified conjugate |
| 5 | Fab 15 non-reduced |
| 6 | Fab 15 reduced |
| 7 | Fab 15 conjugation mix |
| 8 | Fab 15 purified conjugate |
| 9 | Fab 16 non-reduced |
| 10 | Fab 16 reduced |
| 11 | Fab 16 conjugation mix |
| 12 | Fab 16 purified conjugate |
| 13 | Fab 17 non-reduced |
| 14 | Fab 17 reduced |
| 15 | Fab 17 conjugation mix |
| 16 | Fab 17 purified conjugate |
| 17 | Fab 18 non-reduced |
| 18 | Fab 18 reduced |
| 19 | Fab 18 conjugation mix |
| 20 | Fab 18 purified conjugate |
| 21 | Fab 19 non-reduced |
| 22 | Fab 19 reduced |
| 23 | Fab 19 conjugation mix |
| 24 | Fab 19 purified conjugate |
| M | Marker |

FIG. 8 shows SDS-PAGE analysis of the conjugated and unconjugated constructs Fab 20, Fab 21 and Fab 22 under various conditions (as set forth in Table 3).

TABLE 3
Lane contents for FIG. 8

| Lane | Sample |
|---|---|
| M | Ladder |
| 1 | Fab 20 reduced |
| 2 | Fab 20 non-reduced |
| 3 | Fab 20 conjugation mix |
| 4 | Fab 20 purified conjugate |
| 5 | Fab 21 non-reduced |
| 6 | Fab 21 reduced |
| 7 | Fab 21 conjugation mix |
| 8 | Fab 21 purified conjugate |
| 9 | Fab 22 non-reduced |
| 10 | Fab 22 reduced |
| 11 | Fab 22 conjugation mix |
| 12 | Fab 22 purified conjugate |

FIG. 9 shows SDS-PAGE analysis of the conjugated to mal-PEG-DPSE and unconjugated constructs Fabs 11-22 under various conditions (as set forth in Table 4).

TABLE 4
Lane contents for FIG. 9

| Lane | Sample |
|---|---|
| M | Ladder |
| 1 | Fab 11 reduced |
| 2 | Fab 11 reduced, purified conjugate |
| 3 | Fab 15 reduced |
| 4 | Fab 15 reduced, purified conjugate |
| 5 | Fab 16 reduced |
| 6 | Fab 16 reduced, purified conjugate |
| 7 | Fab 17 reduced |
| 8 | Fab 17 reduced, purified conjugate |
| 9 | Fab 18 reduced |
| 10 | Fab 18 reduced, purified conjugate |
| 11 | Fab 19 reduced |
| 12 | Fab 19 reduced, purified conjugate |
| 13 | Fab 12 reduced |
| 14 | Fab 12 reduced, purified conjugate |
| 15 | Fab 13 reduced |
| 16 | Fab 13 reduced, purified conjugate |
| 17 | Fab 14 reduced |
| 18 | Fab 14 reduced, purified conjugate |
| 19 | Fab 20 reduced |
| 20 | Fab 20 reduced, purified conjugate |
| 21 | Fab 21 reduced |
| 22 | Fab 21 reduced, purified conjugate |
| 23 | Fab 22 reduced |
| 24 | Fab 22 reduced, purified conjugate |
| M | Marker |

FIG. 10 shows SDS-PAGE analysis of engineered Fabs conjugated to doxorubicin liposomes as well as unconjugated constructs Fabs 11-22 under various conditions (as set forth in Table 5).

TABLE 5
Lane contents for FIG. 10

| Lane | Sample |
|---|---|
| M | Ladder |
| 1 | Fab 11 non-reduced |
| 2 | Fab 11 non-reduced, purified conjugate |
| 3 | Fab 15 non-reduced |
| 4 | Fab 15 non-reduced, purified conjugate |
| 5 | Fab 16 non-reduced |
| 6 | Fab 16 non-reduced, purified conjugate |
| 7 | Fab 17 non-reduced |
| 8 | Fab 17 non-reduced, purified conjugate |
| 9 | Fab 18 non-reduced |
| 10 | Fab 18 non-reduced, purified conjugate |
| 11 | Fab 19 non-reduced |
| 12 | Fab 19 non-reduced, purified conjugate |
| 13 | 1 μg BSA standard |
| 14 | 0.75 μg BSA standard |
| 15 | 0.5 μg BSA standard |
| 16 | 0.25 μg BSA standard |
| 17 | Fab 11 non-reduced |
| 18 | Fab 11 non-reduced purified conjugate |
| 19 | Fab 12 non-reduced |
| 20 | Fab 12 non-reduced, purified conjugate |
| 21 | Fab 13 non-reduced |
| 22 | Fab 13 non-reduced, purified conjugate |
| 23 | Fab 14 non-reduced |
| 24 | Fab 14 non-reduced, purified conjugate |
| 25 | Fab 20 non-reduced |
| 26 | Fab 20 non-reduced, purified conjugate |
| 27 | Fab 21 non-reduced |
| 28 | Fab 21 non-reduced, purified conjugate |
| 29 | Fab 22 non-reduced |
| 30 | Fab 22 non-reduced, purified conjugate |

### BRIEF DESCRIPTION OF THE SEQUENCES

The amino acid (“aa”) sequences referred to herein and listed in the sequence listing are identified below.
SEQ ID NO:1 Fab 1 IgG1 wild-type heavy chain
SEQ ID NO:2 Fab 1, Fab 3, Fab 9 kappa wild-type light chain
SEQ ID NO:3 Fab 2, Fab 6 IgG1(C233S) heavy chain
SEQ ID NO:4 Fab 2, Fab 4, Fab 10 kappa (C214S) light chain
SEQ ID NO:5 Fab 3 IgG2 wild-type heavy chain
SEQ ID NO:6 Fab 4 IgG2 (C127S) heavy chain
SEQ ID NO:7 Fab 5 IgG1 (G44C, C233S) heavy chain
SEQ ID NO:8 Fab 5 kappa (G100C, C214S) light chain
SEQ ID NO:9 Fab 6 kappa (P80C, 1106V, S171C, C214S) light chain
SEQ ID NO:10 Fab 7 IgG1 (F174C+C233S) heavy chain
SEQ ID NO:11 Fab 7 kappa (S176C, C214S) light chain
SEQ ID NO:12 Fab 8 IgG1 (L124C, C233S) heavy chain
SEQ ID NO:13 Fab 8 kappa (F118C, C214S) light chain
SEQ ID NO:14 Fab 9 IgG4 wild-type heavy chain
SEQ ID NO:15 Fab 10 IgG4 (C217S) heavy chain
SEQ ID NO:16 anti-EphA2, Fab 11 IgG1 (“wildtype”) heavy chain
SEQ ID NO:17 anti-EphA2, Fab 11 lambda (“wildtype”) light chain
SEQ ID NO:18 anti-EphA2, Fab 12 IgG1 (G44C, C233S) heavy chain
SEQ ID NO:19 anti-EphA2, Fab 12 lambda (G100C, C214S) light chain
SEQ ID NO:20 anti-EphA2, Fab 13 IgG1 (F174C, C233S) heavy chain
SEQ ID NO:21 anti-EphA2, Fab 13 lambda (S176C, C214S) light chain
SEQ ID NO:22 anti-EphA2, Fab 14 IgG1 (G44C, F174C, C233S) heavy chain
SEQ ID NO:23 anti-EphA2, Fab 14 lambda (G100C, S176C, C214S) light chain
SEQ ID NO:24 anti-EphA2, Fab 15 IgG1 (H172E, F174C, C233S) heavy chain
SEQ ID NO:25 anti-EphA2, Fab 15 lambda (T162D, S176C, C214S) light chain
SEQ ID NO:26 anti-EphA2, Fab 16 IgG1 (H172F, F174C, C233S) heavy chain
SEQ ID NO:27 anti-EphA2, Fab 16 lambda (T162L, S174V, S176C, C214S) light chain
SEQ ID NO:28 anti-EphA2, Fab 17 IgG1 (G44L, F174C, C233S) heavy chain
SEQ ID NO:29 anti-EphA2, Fab 17 lambda (G100L, S176C, C214S) light chain
SEQ ID NO:30 anti-EphA2, Fab 18 IgG1 (G44L, H172E, F174C, C233S) heavy chain
SEQ ID NO:31 anti-EphA2, Fab 18 lambda (G100L, T162D, S176C, C214S) light chain
SEQ ID NO:32 anti-EphA2, Fab 19 IgG1 (G44L, H172F, F174C, C233S) heavy chain
SEQ ID NO:33 anti-EphA2, Fab 19 lambda (G100L, T162L, S174V, S176C, C214S) light chain
SEQ ID NO:34 Fab 20 IgG1 (H172E, F174C, C233S) heavy chain
SEQ ID NO:35 Fab 20 kappa (S162D, S176C, C214S) light chain
SEQ ID NO:36 Fab 21 IgG1 (H172F, F174C, C233S) heavy chain
SEQ ID NO:37 Fab 21 kappa (S162L, S174V, S176C, C214S) light chain
SEQ ID NO:38 Fab 22 IgG1 (G44L, F174C, C233S) heavy chain
SEQ ID NO:39 Fab 22 kappa (G100L, S176C, C214S) light chain
SEQ ID NO:40 Fab 23 IgG1 (G44L, H172E, F174C, C233S:) heavy chain
SEQ ID NO:41 Fab 23 kappa (G100L, S162D, S176C, C214S) light chain
SEQ ID NO:42 Fab 24 IgG1 (G44L, H172F, F174C, C233S) heavy chain
SEQ ID NO:43 Fab 24 kappa (G100L, S162L, S174V, S176C, C214S) light chain
SEQ ID NO:44 CH1 IgG1 C-terminus appended sequence
SEQ ID NO:45 CH1 IgG2 C-terminus appended sequence
SEQ ID NO:46 CH1 IgG4 C-terminus appended sequence
SEQ ID NO:47 Human EphA2 with C-terminus appended hexahistidine tag

### DETAILED DESCRIPTION

Provided herein are novel disulfide-stabilized Fabs. These engineered Fabs lack at least one native disulfide bond, and contain at least one introduced, engineered (i.e., not naturally occurring) disulfide bond. The Fabs may have a naturally occurring or an engineered cysteine residue within 10 amino acid residues from the C-terminus of the Fab heavy chain (i.e., within or C-terminal to the CH1), which residue may be embedded within an engineered C-terminal or juxta-C-terminal linker sequence (e.g., of from 2 to 20 amino acids in length). Such engineered Fabs allow for site-specific conjugation of an effector moiety the C-terminal cysteine of the heavy chain without denaturing or disrupting (e.g., by attaching to one of the cysteines of) Fab disulfide bonds.

### Definitions

“aa” indicates amino acid.

“Binding strength” refers to the strength of a binding interaction and includes both the actual binding affinity as well as the apparent binding affinity. The actual binding affinity is a ratio of the association rate over the disassociation rate. The apparent affinity can include, for example, the additional binding strength (avidity) resulting from a polyvalent interaction. Dissociation constant (K_{d}), is typically the reciprocal of the binding affinity.

“CH1” or “C_{H}1” refers to the immunoglobulin heavy chain constant region spanning positions 114-223 (located between the VH and the hinge). A CH1 can be a naturally occurring (“native”) CH1 or an engineered variant of a naturally occurring CH1 (in which one or more amino acids have been substituted, added or deleted), provided that the engineered CH1 has a desired biological property (e.g., when incorporated into a Fab it does not abrogate functional immunospecific antigen binding as compared to a Fab comprising the CH1 from which the engineered CH1 was derived).

“CL” or “C_{L}” refers to the immunoglobulin light chain constant region that spans about positions 107A-216 is located C-terminally to the VH. It. A CL can be a naturally occurring CL, or a naturally occurring CL in which one or more amino acids have been substituted, added or deleted, provided that the CL has a desired biological property (e.g., when incorporated into a Fab it does not abrogate functional immunospecific antigen binding as compared to a Fab comprising the CL from which the engineered CL was derived). A CL may or may not comprise a C-terminal lysine.

“Conservative substitution” refers to the replacement of one or more aa residues in a protein or a peptide with, for each particular pre-substitution aa residue, a specific replacement aa that is known to be unlikely to alter either the confirmation or the function of a protein or peptide in which such a particular aa residue is substituted for by such a specific replacement aa. Such conservative substitutions typically involve replacing one aa with another that is similar in charge and/or size to the first aa, and include replacing any of isoleucine (I), valine (V), or leucine (L) for each other, substituting aspartic acid (D) for glutamic acid (E) and vice versa; glutamine (Q) for asparagine (N) and vice versa; and serine (S) for threonine (T) and vice versa. Other substitutions are known in the art to be conservative in particular sequence or structural environments. For example, glycine (G) and alanine (A) can frequently be substituted for each other to yield a conservative substitution, as can be alanine and valine (V). Methionine (M), which is relatively hydrophobic, can frequently conservatively substitute for or be conservatively substituted by leucine or isoleucine, and sometimes valine. Lysine (K) and arginine (R) are frequently interchangeable in locations in which the significant feature of the aa residue is its charge and the differing pK's of these two basic aa residues are not expected to be significant. The effects of such substitutions can be calculated using substitution score matrices such PAM120, PAM-200, and PAM-250.

“Engineered cysteine” means a cysteine that has been introduced into an antibody sequence at a location where a cysteine was not present. Typically the engineered cysteine replaces another amino acid normally found at that position. Cysteines are sometimes engineered as one or more cysteine pairs, e.g., consisting of a cysteine in the heavy chain and a cysteine in the light chain, which heavy chain/light chain cysteine pair allows a disulfide bond to be formed between the heavy and light chains of the antibody fragment.

“Fab” refers to one (or a linked pair of—which format is typically referred to as “F(ab′)₂”) antigen binding antibody fragment(s), each comprising two polypeptide chains: a first chain that comprises a VH and a CH1 and a second chain that comprises a VL and a CL. Fabs were originally obtained as an N-terminal fragment of a full sized antibody cleaved off by treatment with papain. Papain cleavage produces Fabs which comprise a portion of a hinge region that does not include a cysteine that forms a disulfide bond linking two heavy chains, while mild pepsin cleavage of a full sized antibody produces a F(ab′)2 comprising a disulfide bond linking two heavy chains. Recombinantly expressed Fabs can be prepared that are expressed in truncated forms that comprise different portions of a hinge, or lack hinge sequences entirely.

“Hinge” or “hinge region” refers to the flexible portion of a heavy chain located between the CH1 and the CH2. A native hinge is typically about 25 amino acids long.

“Native interchain disulfide bond” refers to an interchain disulfide bond that exists between cysteines in the CH and the CL, each of which cysteines is encoded by a naturally occurring heavy chain or light chain-encoding mRNA. The native interchain cysteines are comprised of a cysteine in the CL and a cysteine in the CH1 that are disulfide linked to each other in naturally occurring antibodies. Such cysteines can be found, e.g., at position 214 of the light chain and 233 of the heavy chain of human IgG1, position 127 of the heavy chain of human IgM, IgE, IgG2, IgG3 and IgG4, and at position 128 of the heavy chain of human IgD and IgA2B.

“Positions,” “position”—all numbered positions set forth herein are numbered according to Kabat et al., “Sequences of proteins of immunological interest” (1991).

“VL” or “V_{L}” refers to a variable region of an immunoglobulin light chain.

“VH” or “V_{H}” refers to a variable region of an immunoglobulin heavy chain.

### Disulfide Stabilized Fabs

Provided herein are Fabs that can be conjugated with moieties, such as effectors (e.g., liposomes), in which a heavy and a light chain of a Fab is linked by at least one engineered interchain disulfide bond that is not a native interchain disulfide bond. The engineered interchain disulfide bond(s) is(are) retained during effector attachment when the effector is attached to an available cysteine, such as one that is further engineered into the molecule, e.g., appended to a heavy or light chain at the C-terminus or near the C-terminus (juxta-C-terminal, i.e., within 10 or 15 amino acid residues of the C-terminus) of the heavy or light chain. Preferred sites for juxta-C-terminal engineered cysteines are at or near the C-terminus of a CH1 or at or near the C-terminus of a CL.

Also provided herein are exemplary engineered Fabs designated as Fab 5, Fab 6, Fab 7, Fab 8, Fab 12, Fab 13, Fab 14, Fab 15, Fab 16, Fab 17, Fab 18, Fab 19, Fab 20, Fab 21, Fab 22, Fab 23 and Fab 24. Table 6 shows an indication of the engineered cysteines and the SEQ ID NOs for an exemplary heavy and light chain sequences (amino acid sequences are shown in Table 7, where engineered cysteines are in boldface and underlined, substituted cysteines are in boldface and italics, and double underlines indicate additional substituted residues).

TABLE 6
Exemplary anti-EphA2 Engineered Fabs

| | Engineered cysteine pairs | |
|---|---|---|
| | (Heavy chain position- | SEQ ID NOs |
| Fab | Light chain position) | (Heavy chain, Light chain) |
| 11 | n/a | 16, 17 |
| 12 | 44-100 | 18, 19 |
| 13 | 174-176 | 20, 21 |
| 14 | 44-100 | 22, 23 |
| | 174-176 | |
| 15 | 174-176 | 24, 25 |
| 16 | 174-176 | 26, 27 |
| 17 | 174-176 | 28, 29 |
| 18 | 174-176 | 30, 31 |
| 19 | 174-176 | 32, 33 |

TABLE 7
Polypeptide sequences

Fab 1 Heavy chain (SEQ ID NO: 1)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCAA
Fab 1 Light chain (SEQ ID NO: 2)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Fab 2 Heavy chain (SEQ ID NO: 3)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKS [symbol] DKTHTCAA
Fab 2 Light chain (SEQ ID NO: 4)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE [symbol]
Fab 3 Heavy chain (SEQ ID NO: 5)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPCSRSTSESTAALGCLVKDYFPEPVTVSWNSGALTSGVHTFPAVL
QSSGLYSLSSVVTVPSSNFGTQTYTCNVDHKPSNTKVDKTVERKCAA
Fab 3 Light chain (SEQ ID NO: 2)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Fab 4 Heavy chain (SEQ ID NO: 6)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAP [symbol] SRSTSESTAALGCLVKDYFPEPVTVSWNSGALTSGVHTFPAVL
QSSGLYSLSSVVTVPSSNFGTQTYTCNVDHKPSNTKVDKTVERKCAA
Fab 4 Light chain (SEQ ID NO: 4)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE [symbol]
Fab 5 Heavy chain (SEQ ID NO: 7)
QVQLVQSGAEVKKPGSSVKVSCKASGGTFSSYAISWVRQAPGQCLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKS [symbol] DKTHTCAA
Fab 5 Light chain (SEQ ID NO: 8)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGCGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE [symbol]
Fab 6 Heavy chain (SEQ ID NO: 3)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKS [symbol] DKTHTCAA
Fab 6 Light chain (SEQ ID NO: 9)
AIQLTQSPSTLSASVGDRVTITCRASQSISSWLAWYQQKPGKAPKLLIYKASSLESGVPSRF
SGSGSGTEFTLTISSLQCDDFATYYCQQYNSYPYTFGQGTKLEVKRTVAAPSVFIFPPSDEQ
LKSGTASVVCLLNNFYPREAKVQWKVDNALQSGNSQESVTEQDSKDCTYSLSSTLTLSKADY
EKHKVYACEVTHQGLSSPVTKSFNRGE [symbol]
Fab 7 Heavy chain (SEQ ID NO: 10)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVHTCPAVL
QSSGLYSLSSVVTVPSSSLGTQTYICNVNHKPSNTKVDKKVEPKS [symbol] DKTHTCAA
Fab 7 Light chain (SEQ ID NO: 11)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLCSTLTLSKADY
EKHKVYACEVTHQGLSSPVTKSFNRGE [symbol]
Fab 8 Heavy chain (SEQ ID NO: 12)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPCAPSSKSTSGGTAALGCLVKDYFPEPVTVSWNSGALTSGVHTFPAVL
QSSGLYSLSSVVTVPSSSLGTQTYICNVNHKPSNTKVDKKVEPKS [symbol] DKTHTCAA
Fab 8 Light chain (SEQ ID NO: 13)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFICPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE [symbol]
Fab 9 Heavy chain (SEQ ID NO: 14)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGCAA
Fab 9 Light chain (SEQ ID NO: 2)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Fab 10 Heavy chain (SEQ ID NO: 15)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAP [symbol] SRSTSESTAALGCLVKDYFPEPVTVSWNSGALTSGVHTFPAVL
QSSGLYSLSSVVTVPSSSLGTKTYTCNVDHKPSNTKVDKRVESKYGCAA
Fab 10 Light chain (SEQ ID NO: 4)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGE [symbol]
Fab 11 Heavy chain (SEQ ID NO: 16)
QVQLVQSGGGLVQPGGSLRLSCAASGFTFSSYAMHWVRQAPGKGLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCAA
Fab 11 Light chain (SEQ ID NO: 17)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGGGTKLTVLGQPKAAPSVTLFPPS
SEELQANKATLVCLVSDFYPGAVTVAWKADGSPVKVGVETTKPSKQSNNKYAASSYLSLTPE
QWKSHRSYSCRVTHEGSTVEKTVAPAECS
Fab 12 Heavy chain (SEQ ID NO: 18)
QVQLVQSGGGLVQPGGSLRLSCAASGFTFSSYAMHWVRQAPGKCLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKS [symbol] DKTHTCAA
Fab 12 Light chain (SEQ ID NO: 19)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGCGTKLTVLGQPKAAPSVTLFPPS
SEELQANKATLVCLVSDFYPGAVTVAWKADGSPVKVGVETTKPSKQSNNKYAASSYLSLTPE
QWKSHRSYSCRVTHEGSTVEKTVAPAE [symbol] S
Fab 13 Heavy chain (SEQ ID NO: 20)
QVQLVQSGGGLVQPGGSLRLSCAASGFTFSSYAMHWVRQAPGKGLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVHTCPAVLQSSGLYSLS
SVVTVPSSSLGTQTYICNVNHKPSNTKVDKKVEPKS [symbol] DKTHTCAA
Fab 13 Light chain (SEQ ID NO: 21)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGGGTKLTVLGQPKAAPSVTLFPPS
SEELQANKATLVCLVSDFYPGAVTVAWKADGSPVKVGVETTKPSKQSNNKYAACSYLSLTPE
QWKSHRSYSCRVTHEGSTVEKTVAPAE [symbol] S
Fab 14 Heavy chain (SEQ ID NO: 22)
QVQLVQSGGGLVQPGGSLRLSCAASGFTFSSYAMHWVRQAPGKCLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVHTCPAVLQSSGLYSLS
SVVTVPSSSLGTQTYICNVNHKPSNTKVDKKVEPKS [symbol] DKTHTCAA
Fab 14 Light chain (SEQ ID NO: 23)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGCGTKLTVLGQPKAAPSVTLFPPS
SEELQANKATLVCLVSDFYPGAVTVAWKADGSPVKVGVETTKPSKQSNNKYAACSYLSLTPE
QWKSHRSYSCRVTHEGSTVEKTVAPAE [symbol] S
Fab 15 Heavy chain (SEQ ID NO: 24)
QVQLVQSGGGLVQPGGSLRLSCAASGFTFSSYAMHWVRQAPGKGLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVETCPAVLQSSGLYSLS
SVVTVPSSSLGTQTYICNVNHKPSNTKVDKKVEPKS [symbol] DKTHTCAA
Fab 15 Light chain (SEQ ID NO: 25)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGGGTKLTVLGQPKAAPSVTLFPPS
SEELQANKATLVCLVSDFYPGAVTVAWKADGSPVKVGVEDTKPSKQSNNKYAACSYLSLTPE
QWKSHRSYSCRVTHEGSTVEKTVAPAE [symbol] S
Fab 16 Heavy chain (SEQ ID NO: 26)
QVQLVQSGGGLVQPGGSLRLSCAASGFTFSSYAMHWVRQAPGKGLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVFTCPAVLQSSGLYSLS
SVVTVPSSSLGTQTYICNVNHKPSNTKVDKKVEPKS [symbol] DKTHTCAA
Fab 16 Light chain (SEQ ID NO: 27)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGGGTKLTVLGQPKAAPSVTLFPPS
SEELQANKATLVCLVSDFYPGAVTVAWKADGSPVKVGVELTKPSKQSNNKYVACSYLSLTPE
QWKSHRSYSCRVTHEGSTVEKTVAPAE [symbol] S
Fab 17 Heavy chain (SEQ ID NO: 28)
QVQLVQSGGGLVQPGGSLRLSCAASGFTFSSYAMHWVRQAPGKLLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVHTCPAVLQSSGLYSLS
SVVTVPSSSLGTQTYICNVNHKPSNTKVDKKVEPKS [symbol] DKTHTCAA
Fab 17 Light chain (SEQ ID NO: 29)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGLGTKLTVLGQPKAAPSVTLFPPS
SEELQANKATLVCLVSDFYPGAVTVAWKADGSPVKVGVETTKPSKQSNNKYAACSYLSLTPE
QWKSHRSYSCRVTHEGSTVEKTVAPAE [symbol] S
Fab 18 Heavy chain (SEQ ID NO: 30)
QVQLVQSGGGLVQPGGSLRLSCAASGFTFSSYAMHWVRQAPGKLLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVETCPAVLQSSGLYSLS
SVVTVPSSSLGTQTYICNVNHKPSNTKVDKKVEPKS [symbol] DKTHTCAA
Fab 18 Light chain (SEQ ID NO: 31)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGLGTKLTVLGQPKAAPSVTLFPPS
SEELQANKATLVCLVSDFYPGAVTVAWKADGSPVKVGVEDTKPSKQSNNKYAACSYLSLTPE
QWKSHRSYSCRVTHEGSTVEKTVAPAE [symbol] S
Fab 19 Heavy chain (SEQ ID NO: 32)
QVQLVQSGGGLVQPGGSLRLSCAASGFTFSSYAMHWVRQAPGKLLEWVTVISPDGHNTYYAD
SVKGRFTISRDNSKNTLYLQMNSLRAEDTAVYYCARASVGATGPFDIWGQGTLVTVSSASTK
GPSVFPLAPSSKSTSGGTAALGCLVKDYFPEPVTVSWNSGALTSGVFTCPAVLQSSGLYSLS
SVVTVPSSSLGTQTYICNVNHKPSNTKVDKKVEPKS [symbol] DKTHTCAA
Fab 19 Light chain (SEQ ID NO: 33)
SSELTQPPSVSVAPGQTVTITCQGDSLRSYYASWYQQKPGTAPKLLIYGENNRPSGVPDRFS
GSSSGTSASLTITGAQAEDEADYYCNSRDSSGTHLTVFGLGTKLTVLGQPKAAPSVTLFPPS
SEELQANKATLVCLVSDFYPGAVTVAWKADGSPVKVGVELTKPSKQSNNKYVACSYLSLTPE
QWKSHRSYSCRVTHEGSTVEKTVAPAE [symbol] S
Fab 20 Heavy chain (SEQ ID NO: 34)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVETCPAVL
QSSGLYSLSSVVTVPSSSLGTQTYICNVNHKPSNTKVDKKVEPKS [symbol] DKTHTCAA
Fab 20 Light chain (SEQ ID NO: 35)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQEDVTEQDSKDSTYSLCSTLTLSKADY
EKHKVYACEVTHQGLSSPVTKSFNRGE [symbol]
Fab 21 Heavy chain (SEQ ID NO: 36)
QVQLVQSGAEVKKPGSSVKVSCKASGGTFSSYAISWVRQAPGQGLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVFTCPAVL
QSSGLYSLSSVVTVPSSSLGTQTYICNVNHKPSNTKVDKKVEPKS [symbol] DKTHTCAA
Fab 21 Light chain (SEQ ID NO: 37)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQELVTEQDSKDSTYVLCSTLTLSKADY
EKHKVYACEVTHQGLSSPVTKSFNRGE [symbol]
Fab 22 Heavy chain (SEQ ID NO: 38)
QVQLVQSGAEVKKPGSSVKVSCKASGGTFSSYAISWVRQAPGQLLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVHTCPAVL
QSSGLYSLSSVVTVPSSSLGTQTYICNVNHKPSNTKVDKKVEPKS [symbol] DKTHTCAA
Fab 22 Light chain (SEQ ID NO: 39)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQLTKLEIKRTVAAPSVFIFPPSDEQ
LKSGTASVVCLLNNFYPREAKVQWKVDNALQSGNSQESVTEQDSKDSTYSLCSTLTLSKADY
EKHKVYACEVTHQGLSSPVTKSFNRGE [symbol]
Fab 23 Heavy chain (SEQ ID NO: 40)
QVQLVQSGAEVKKPGSSVKVSCKASGGTFSSYAISWVRQAPGQLLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVETCPAVL
QSSGLYSLSSVVTVPSSSLGTQTYICNVNHKPSNTKVDKKVEPKS [symbol] DKTHTCAA
Fab 23 Light chain (SEQ ID NO: 41)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQLTKLEIKRTVAAPSVFIFPPSDEQ
LKSGTASVVCLLNNFYPREAKVQWKVDNALQSGNSQEDVTEQDSKDSTYSLCSTLTLSKADY
EKHKVYACEVTHQGLSSPVTKSFNRGE [symbol]
Fab 24 Heavy chain (SEQ ID NO: 42)
QVQLVQSGAEVKKPGSSVKVSCKASGGTFSSYAISWVRQAPGQLLEWMGGIIPIFGTANYAQ
KFQGRVTITADESTSTAYMELSSLRSEDTAVYYCARDPKRDDYIWGSYRPQYAFDIWGQGTM
VTVSSASTKGPSVFPLAPSSKSTSGGTAALGCLVKDYFPEPVTVSWNSGALTSGVFTCPAVL
QSSGLYSLSSVVTVPSSSLGTQTYICNVNHKPSNTKVDKKVEPKS [symbol] DKTHTCAA
Fab 24 Light chain (SEQ ID NO: 43)
AIQLTQSPSTLSASVGDRVTITCRASQSISSWLAWYQQKPGKAPKLLIYKASSLESGVPSRF
SGSGSGTEFTLTISSLQPDDFATYYCQQYNSYPYTFGQLTKLEIKRTVAAPSVFIFPPSDEQ
LKSGTASVVCLLNNFYPREAKVQWKVDNALQSGNSQELVTEQDSKDSTYVLCSTLTLSKADY
EKHKVYACEVTHQGLSSPVTKSFNRGE [symbol]
Linker IgG1 (SEQ ID NO: 44)
DKTHTCAA
Linker IgG2 (SEQ ID NO: 45)
ERKCAA
Linker IgG4 (SEQ ID NO: 46)
ESKYGCAA
Recombinant, Human EphA2 with C-terminal hexahistidine
appended (SEQ ID NO: 47)
QGKEVVLLDFAAAGGELGWLTHPYGKGWDLMQNIMNDMPIYMYSVCNVMSGDQDNWLRTNWV
YRGEAERIFIELKFTVRDCNSFPGGASSCKETFNLYYAESDLDYGTNFQKRLFTKIDTIAPD
EITVSSDFEARHVKLNVEERSVGPLTRKGFYLAFQDIGACVALLSVRVYYKKCPELLQGLAH
FPETIAGSDAPSLATVAGTCVDHAVVPPGGEEPRMHCAVDGEWLVPIGQCLCQAGYEKVEDA
CQACSPGFFKFEASESPCLECPEHTLPSPEGATSCECEEGFFRAPQDPASMPCTRPPSAPHY
LTAVGMGAKVELRWTPPQDSGGREDIVYSVTCEQCWPESGECGPCEASVRYSEPPHGLTRTS
VTVSDLEPHMNYTFTVEARNGVSGLVTSRSFRTASVSINQTEPPKVRLEGRSTTSLSVSWSI
PPPQQSRVWKYEVTYRKKGDSNSYNVRRTEGFSVTLDDLAPDTTYLVQVQALTQEGQGAGSK
VHEFQTLSPEGSGNHHHHHH

Fab 5 and Fab 12 fragments are characterized in that

(a) a native inter-chain disulfide bond between native inter-chain cysteines in the heavy (CH1) and light (CL) chain constant regions is absent, and
(b) the heavy chain (VH) and light chain (VL) variable regions are linked by an inter-chain disulfide bond between a pair of engineered cysteines, one in the light chain variable (VL) region and the other in the heavy chain variable (VH) region, wherein the position of the pair of engineered cysteines is position 44 of the heavy chain and position 100 of the light chain.

Fab 7 and Fab 13 fragments are characterized in that

(a) a native inter-chain disulfide bond between native inter-chain cysteines in the heavy (CH1) and light (CL) chain constant regions is absent, and
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain.

Fab 14 fragments are characterized in that

(a) a native inter-chain disulfide bond between native inter-chain cysteines in the heavy (CH1) and light (CL) chain constant regions is absent, and
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain,
and further characterized in that the heavy chain (VH) and light chain (VL) variable regions are linked by an second inter-chain disulfide bond between a second pair of engineered cysteines, one in the light chain variable (VL) region and the other in the heavy chain variable (VH) region, wherein the position of the second pair of engineered cysteines is position 44 of the heavy chain and position 100 of the light chain.

Fab 15 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is glutamic acid at heavy chain constant (CH1) region position 172 and aspartic acid at light chain constant (CL) region position 162.

Fab 16 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (ii) phenylalanine at heavy chain constant region (CH1) position 172 and leucine at light chain constant (CL) region position 162; and (ii) valine at light chain (CL) position 174.

Fab 17 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is leucine at heavy chain variable (VH) region position 44 and leucine at light chain variable (VL) region position 100.

Fab 18 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (i) glutamic acid at heavy chain constant (CH1) region position 172 and aspartic acid at light chain constant (CL) region position 162; and (ii) leucine at heavy chain variable (VH) region position 44 and leucine at light chain variable (VL) region position 100.

Fab 19 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (i) phenylalanine at heavy chain constant (CH1) region position 172 and leucine acid at light chain constant (CL) region position 162; (ii) leucine at heavy chain variable (VH) region position 44 and leucine at light chain variable (VL) region position 100; and (iii) valine at light chain constant (CL) region position 174.

Fab 20 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (i) glutamic acid at heavy chain constant (CH1) region position 172 and aspartic acid at light chain constant (CL) region position 162; and (ii) leucine at heavy chain variable (VH) region position 44 and leucine at light chain variable (VL) region position 100.

Fab 21 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (i) glutamic acid at heavy chain constant (CH1) region position 172 and aspartic acid at light chain constant (CL) region position 162; and (ii) valine at light chain constant (CL) region position 174.

Fab 22 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is leucine at heavy chain variable (VH) region position 44 and leucine at light chain variable (VL) region position 100.

Fab 23 fragments are characterized in that

(a) a native inter-chain disulfide bond between native inter-chain cysteines in the heavy (CH1) and light (CL) chain constant regions is absent,
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (i) glutamic acid at heavy chain constant (CH1) region position 172 and aspartic acid at light chain constant (CL) region position 162; and (ii) leucine at heavy chain variable (VH) region position 44 and leucine at light chain variable (VL) region position 100.

Fab24 fragments are characterized in that

(a) a native inter-chain disulfide bond between native inter-chain cysteines in the heavy (CH1) and light (CL) chain constant regions is absent, and
(b) the heavy chain (CH1) and light chain (CL) constant regions are linked by an inter-chain disulfide bond between a pair of engineered cysteines, one in the light chain constant (CL) region and the other in the heavy chain constant (CH1) region, wherein the position of the pair of engineered cysteines is position 174 of the heavy chain and position 176 of the light chain, and wherein
(c) there is (i) phenylalanine at heavy chain constant (CH1) region position 172 and leucine acid at light chain constant (CL) region position 162; (ii) leucine at heavy chain variable (VH) region position 44 and leucine at light chain variable (VL) region position 100; and (iii) valine at light chain constant (CL) region position 174

Fab 1, Fab 2, Fab 5, Fab 6, Fab 7, Fab 8, Fab 9, Fab 10, Fab 11, Fab 12, Fab 13, Fab 14, Fab 15, Fab 16, Fab 17, Fab 18, Fab 19, Fab 20, Fab 21, Fab 22, Fab 23 and Fab 24 (i.e., Fabs 1 through 24), can optionally have at least one amino acid appended to a terminus, for example, at the C-terminus of the CHI. In some embodiments, the appended at least one amino acid is SEQ ID NO:44. In other embodiments, the appended at least one amino acid comprises or consists of SEQ ID NO:45 or SEQ ID NO:46.

Provided that the substitutions as specified for each numbered Fab are retained, thermostability at ≥60° C. is maintained, and detectable immunospecific binding is preserved, additional Fabs provided herein include those that are 70%, 75%, 80%, 85%, 90%, 95%, 96%, 97%, 98%, or 99 identical to Fabs 1 through 24, which % identities may be achieved via conservative substitutions to Fabs 1-Fab 24.

### Nucleic Acid, Expression Vectors and Host Cells

The antibodies described herein can be produced by recombinant means. Methods for recombinant production comprise protein expression in cells (e.g., cultured cells) with subsequent isolation of the antibody and usually purification to a pharmaceutically acceptable purity. For the expression of the antibodies in a host cell, nucleic acids encoding the respective polypeptides, e.g., light and heavy chains, are inserted into expression vectors by standard methods that result in functional expression constructs. Expression is performed in appropriate host cells, e.g., CHO cells, NSO cells, SP2/0 cells, HEK293 cells, COS cells, PER.C6 cells, yeast, or E. coli cells, and the protein is recovered from the cells (supernatant or cells after lysis).

Antibodies can be suitably separated from culture medium or cell homogenates by conventional protein purification procedures, for example, chromatographic methods including size exclusion chromatography, protein A or protein G affinity chromatography, ion exchange chromatography (e.g., cation exchange (carboxymethyl resins), anion exchange (amino ethyl resins) and mixed-mode exchange) and metal chelate affinity chromatography (e.g., with Ni(II)- and Cu(II)-affinity material), thiophilic adsorption (e.g., with beta-mercaptoethanol or other SH ligands), hydrophobic interaction or aromatic adsorption chromatography (e.g., with phenyl-sepharose, aza-arenophilic resins, or m-aminophenylboronic acid. Other separation methods include electrophoretic methods such as gel electrophoresis and capillary electrophoresis or dialysis. DNAs and RNAs encoding the antibodies are readily isolated and sequenced using conventional procedures.

### Fabs

Fabs disclosed herein include Fabs, Fab's, F(ab′)₂s or truncated Fabs, e.g., as described in US Patent Pub No. 2007-0059301.

Fabs for use as described herein may possess native or modified hinges. The native hinge region is the hinge region normally associated with the CH1 of the parental antibody molecule. A modified hinge region is any hinge that differs in length and/or composition from the native hinge region. Such hinges can include hinge regions from any suitable species, such as human, mouse, rat, rabbit, pig, hamster, camel, llama or goat hinge regions. Other modified hinge regions may comprise a complete hinge region derived from an antibody of a different class or subclass from that of the CH1. Thus, for instance, a CH1 of class γ1 can be attached to a hinge region of class γ4. Alternatively, the modified hinge region may comprise part of a natural hinge or a repeating unit in which each unit in the repeat is derived from a natural hinge region. In a further alternative, the natural hinge region can be altered by converting one or more cysteine or other residues into neutral residues, such as alanine, or by converting suitably placed residues into cysteine residues. By such means the number of cysteine residues in the hinge region can be increased or decreased. In addition, other characteristics of the hinge can be controlled, such as the distance of the hinge cysteine(s) from the light chain interchain cysteine, the distance between the cysteines of the hinge and the composition of other amino acids in the hinge that may affect properties of the hinge such as flexibility, e.g., glycines, can be incorporated into the hinge to increase rotational flexibility or prolines, can be incorporated to reduce flexibility. Alternatively, combinations of charged or hydrophobic residues can be incorporated into the hinge to confer multimerization properties. Other modified hinge regions can be entirely synthetic and can be designed to possess desired properties such as length, composition and flexibility. A number of modified hinge regions have already been described for example, in U.S. Pat. No. 5,677,425, WO9915549, WO9825971 and WO2005003171.

The antibody starting material can be derived from any antibody isotype including for example IgG, IgM, IgA, IgD and IgE and subclasses thereof including for example IgG1, IgG2, IgG3 and IgG4. The starting material can be obtained from any species including for example mouse, rat, rabbit, pig, hamster, camel, llama, goat or, preferably, human. Parts of the antibody can be obtained from more than one species, for example, the antibody can be chimeric. In one example, the constant regions are from one species and the variable regions are from another.

Methods for creating and manufacturing recombinant antibody fragments are well known (see, e.g., U.S. Pat. Nos. 4,816,397; 6,331,415; 5,585,089; and WO91/09967 and WO 92/02551.

The Fab will in general be capable of immunospecifically binding to an antigen. The antigen can be any cell-associated antigen, for example, a cell surface antigen on cells (e.g., human cells) such as T-cells, endothelial cells or tumor cells, or it can be an extracellular matrix antigen or a soluble antigen. Antigens may also be any medically relevant antigen, such as those antigens upregulated during disease or infection, for example, receptors and/or their corresponding ligands. Particular examples of cell surface antigens include adhesion molecules, for example, integrins such as (31 integrins, e.g., VLA-4, E-selectin, P selectin or L-selectin, CD2, CD3, CD4, CD5, CD7, CD8, CD11a, CD11b, CD18, CD19, CD20, CD23, CD25, CD33, CD38, CD40, CD45, CDW52, CD69, carcinoembryonic antigen (CEA), MUC1, MHC Class I and MHC Class II antigens. Other exemplary antigens include cell surface receptors, e.g., including those for: VEGF, interleukins (such as IL-1, IL-2, IL-3, IL-4, IL-5, IL-6, IL-8, IL-12, IL-16 or IL-17), interferons (such as interferon α, interferon β, or interferon γ, tumor necrosis factor-α, tumor necrosis factor-β), colony stimulating factors (such as G-CSF or GM-CSF), and platelet derived growth factors such as PDGF-α, and PDGF-β. Other receptor antigens include those of insulin-like growth factors (e.g., IGF-R1 and IGF-R2) and ephrins (e.g., ephrin A2), as well as those of the receptors known as EGFR, HER2, ErbB3, ErbB4. Preferred receptor antigens include those that project extracellularly.

### Effectors

In some embodiments, the disclosed Fabs are conjugated to an effector moiety (optionally via a linker). The effector can comprise a drug, e.g., in a lipid conjugate containing a drug.

Where two or more effectors are attached to the Fab these can be identical or different and can be attached to the Fab at different sites or at a single site, by the use of, for example, a branched connecting structure to link two or more effectors to a single site of attachment.

At least one site of effector attachment in the Fab is a cysteine. The cysteine can be reduced to produce a free thiol group suitable for effector attachment. Modified Fabs may therefore be prepared by reacting a Fab as described herein containing at least one reactive cysteine residue with an effector, such as a thiol-selective activated effector.

Fabs can be incorporated into nanoparticles, such as those described in U.S. Pat. Nos. 8,518,963, 8,603,499, 8,603,534, 8,603,535, 8,905,997; 8,110,179, 8,207,290, and 8,546,521. Such nanoparticles can further comprise a therapeutic agent contained within the nanoparticle.

### Methods

Further provided is a method of producing a Fab to which one or more effectors is attached characterized in that a native interchain disulfide bond between the CH1 and the CL is absent and the heavy chain and light chain are linked by an interchain disulfide bond between a pair of engineered cysteines, one in the light chain and the other in the heavy chain, said method comprising: (a) treating a Fab in which the heavy chain and light chain constant regions are linked by an interchain disulfide bond between an engineered cysteine in the light chain and an engineered cysteine in the heavy chain with a reducing agent capable of generating a free thiol group in a cysteine of the heavy and/or light chain constant region and/or, where present, the hinge and (b) reacting the treated fragment with an effector.

Additional effectors can be attached elsewhere in the antibody fragment, in particular the constant regions and/or, where present, the hinge. If there are two or more effectors to be attached to cysteines in the antibody fragment, the effectors can be attached either simultaneously or sequentially by repeating the process. If two or more effectors are attached to cysteines in the antibody fragment they can be attached simultaneously.

The methods provided herein also extend to one or more steps before and/or after the reduction method described above in which further effectors are attached to the antibody fragment using any suitable method as described previously, for example, via other available amino acid side chains such as amino and imino groups.

The reducing agent for use in producing modified antibody fragments is any reducing agent capable of reducing the available cysteines in the antibody fragment to produce free thiols for effector attachment. Suitable reducing agents can be identified by determining the number of free thiols produced after the antibody fragment is treated with the reducing agent. Methods for determining the number of free thiols are well known in the art (see, e.g., Lyons et al., 1990, Protein Engineering, 3, 703). Reducing agents are widely known in the art and include, for example, those described in Singh et al. (1995, Methods in Enzymology, 251, 167-73). Particular examples include thiol based reducing agents such as cysteine (Cys), reduced glutathione (GSH), .β-mercaptoethanol (β-ME), β-mercaptoethylamine (β-MA), dithioerythritol (DTE), and dithiothreitol (DTT).

Other methods for reducing the antibody fragments include using electrolytic methods, such as described in Leach et al. (1965, Div. Protein. Chem., 4, 23-27) and using photoreduction methods such as described in Ellison et al. (2000, Biotechniques, 28 (2), 324-326). The reducing agent can be a non-thiol based reducing agent capable of liberating one or more thiols in an antibody fragment. The non-thiol based reducing agent can be capable of liberating the native interchain thiols in an antibody fragment. Examples of such reducing agents include trialkylphosphine reducing agents (Ruegg U T and Rudinger, J., 1977, Methods in Enzymology, 47, 111-126; Burns J et al., 1991, J. Org. Chem., 56, 2648-2650; Getz et al., 1999, Analytical Biochemistry, 273, 73-80; Han and Han, 1994, Analytical Biochemistry, 220, 5-10; Seitz et al., 1999, Euro. J. Nuclear Medicine, 26, 1265-1273; Cline et al., 2004, Biochemistry, 43, 15195-15203), particular examples of which include tris(2-carboxyethyl)phosphine (TCEP), tris butyl phosphine (TBP), tris-(2-cyanoethyl)phosphine, tris-(3-hydroxypropyl)phosphine (THP) and tris-(2-hydroxyethyl)phosphine. The concentration of reducing agent can be determined empirically, for example, by varying the concentration of reducing agent and measuring the number of free thiols produced. Typically the reducing agent is used in excess over the antibody fragment for example between 2 and 1000 fold molar excess, such as 2, 3, 4, 5, 10, 100 or 1000-fold excess. In one embodiment, the reductant is used at between 2 and 5 mM.

The reactions in steps can generally be performed in a solvent, for example, an aqueous buffer solution such as acetate or phosphate, at around neutral pH, for example around pH 4.5 to around pH 8.5, typically pH 4.5 to 8, suitably pH 6 to 7. The reaction may generally be performed at any suitable temperature, for example between about 5° C. and about 70° C., for example, at room temperature. The solvent can optionally contain a chelating agent such as EDTA, EGTA, CDTA or DTPA. Often the solvent contains EDTA at between 1 and 5 mM, such as 2 mM. Alternatively, or in addition, the solvent can be a chelating buffer such as citric acid, oxalic acid, folic acid, bicine, tricine, tris or ADA. The effector will generally be used in an excess concentration relative to the concentration of the antibody fragment. Typically, the effector is used in between 2 and 100 fold molar excess, such as a 5, 10 or 50 fold molar excess.

Where necessary, the desired product containing the desired number of effectors and retaining the interchain disulfide between the engineered cysteines can be separated from any starting materials or other product generated during the process of attaching an effector by conventional means, for example by chromatography techniques such as ion exchange, size exclusion, protein A, G or L affinity chromatography or hydrophobic interaction chromatography. Accordingly, the methods disclosed herein may optionally further comprise an additional step in which the antibody fragment to which one or more effectors is attached and in which the engineered interchain disulfide is retained is purified.

In another embodiment, lipidic nanoparticles are attached to Fabs by means of a linker molecule. This comprises preparing a lipidic nanoparticle attached to a Fab by means of a linker molecule, the method comprising incubating a lipidic nanoparticle with a Fab (such as Fab 6, Fab 7, Fab 8, Fab 12, Fab 13, Fab 14, Fab 15, Fab 16, Fab 17, Fab 18, Fab 19, Fab 20, Fab 21, Fab 22, Fab 23 and Fab 24), wherein the Fab is conjugated to a linker molecule comprising a hydrophobic domain, and hydrophilic polymer chain terminally attached to the hydrophobic domain, and a chemical group reactive to one or more functional groups on the Fab and attached to the hydrophilic polymer chain at a terminus contralateral to the hydrophobic domain for a time sufficient to permit the hydrophobic domain to become stably associated with the lipidic nanoparticle. Methods related to such preparation of Fabs linked to lipidic nanoparticles via a linker are described in U.S. Pat. No. 6,210,707.

In another embodiment, lipidic nanoparticles are attached to Fab by means of a terminally appended amino acid sequence, such as SEQ ID NO:44. This comprises preparing a lipidic nanoparticle attached to a Fab, the method comprising incubating a Fab (such as Fab 6, Fab 7, Fab 8, Fab 12, Fab 13, Fab 14, Fab 15, Fab 16, Fab 17, Fab 18, Fab 19, Fab 20, Fab 21, Fab 22, Fab 23 and Fab 24), wherein the Fab comprises a terminally appended amino acid sequence comprising primarily amino acids with hydrophilic side chains, which sequence is followed by a lipid modification site with a synthetically appended lipid moiety, with a lipidic nanoparticle for a time sufficient to permit the lipid moiety to become stably associated with the lipidic nanoparticle. Again, methods related to such preparation of Fabs linked to lipidic nanoparticles via a terminally appended amino acid sequence are described in U.S. Pat. No. 6,210,707.

### Assays

In some embodiments, Fabs disclosed herein have increased stability during conjugation of at least one moiety, such as PEG conjugation, when compared to a native, non-modified Fab.

To measure stability of an engineered Fab, the engineered Fab and a control Fab with a native disulfide bond (such as Fab 11) are conjugated to a linker, such as mal-PEG-DSPE using standard techniques (see Examples) and collected. The collected engineered Fab and control Fab are then assayed by non-reducing SDS-PAGE, visualized, and analyzed for the amount of Fab that migrates as reduced protein versus non-reduced protein. Less non-reduced protein (indicating less chain dissociation) indicates greater stability during conjugation. Alternatively, gel filtration can be used to examine for polypeptides that are monomers versus dimers.

In some embodiments, an engineered Fab exhibits binding strength for its target antigen that is no less than 75% of that of a matched native, non-modified Fab. Binding strength can be measured by determining K_{d}, e.g., by use of a surface plasmon resonance assay (e.g., as determined in a BIACORE 3000 instrument (GE Healthcare)), or a cell binding assay, each of which assays is described in Example 3 of U.S. Pat. No. 7,846,440. Alternatively, a biolayer interferometry device (e.g., FortéBIO® Octet®) may be used to determine K_{d}. To measure binding strength of moieties comprising multiple Fabs, where avidity contributes to binding strength, a chaotropic assay can be used in which antigen is bound to a solid substrate and the microparticles are bound to the antigen by the Fabs. The chaotropic reagent can be added to the sample to inhibit the binding of low binding strength antibodies to the antigen during contact with the substrate-bound antigen. Alternatively, the chaotropic agent can be used to wash the substrate after incubation of the sample with the substrate-bound antigen. Low binding strength microparticles are then stripped from the solid phase antigen by the chaotropic reagent. The ratio of the signal in this assay is determined with an anti-human IgG conjugate containing a signal-generating compound in the presence and in the absence of the chaotropic reagent (added either to the sample or used to wash the solid phase antigen) and is proportional to the level of high binding strength IgG present in the sample. Such methods are disclosed in U.S. Pat. No. 7,432,046. Alternatively, biolayer interferometry devices (e.g., fortéBIO®) can be used to measure binding strength.

### Pharmaceutical Compositions

In another aspect, a composition, e.g., a pharmaceutical composition, is provided for treatment of a disease in a patient, as well as methods of use of such a composition for such treatment. The compositions provided herein contain one or more of the Fabs disclosed herein (optionally bound to an effector) formulated with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral administration, e.g., intravenous, intramuscular, subcutaneous, spinal or epidermal administration (e.g., by injection or infusion) and include sterile aqueous solutions or dispersions and sterile powders for the extemporaneous preparation of sterile injectable solutions or dispersions. Saline solutions and aqueous dextrose and glycerol solutions can be employed as liquid carriers, particularly for injectable solutions. The composition, if desired, can also contain minor amounts of wetting or solubility enhancing agents, stabilizers, preservatives, or pH buffering agents. In many cases, it will be useful to include isotonic agents, for example, sodium chloride, sugars, polyalcohols such as mannitol, sorbitol, glycerol, propylene glycol, and liquid polyethylene glycol in the composition. Prolonged absorption of the injectable compositions can be brought about by including in the composition an agent that delays absorption, for example, monostearate salts and gelatin.

Pharmaceutical compositions typically must be sterile and stable under the conditions of manufacture and storage.

### Anti-EphA2 Antibody Fragments

An exemplary set of Fabs provided herein are disulfide stabilized anti-EphA2 Fabs (i.e., Fabs that bind immunospecifically to human EphA2). Exemplary Fabs include Fab 12 (SEQ ID NOs:18 and 19), Fab 13 (SEQ ID NOs:20 and 21), Fab 14 (SEQ ID NOs:22 and 23), Fab 15 (SEQ ID NOs:24 and 25), Fab 16 (SEQ ID NOs:26 and 27), Fab 17 (SEQ ID NOs:28 and 29), Fab 18 (SEQ ID NOs:30 and 31), and Fab 19 (SEQ ID NO:32 and 33) as shown in Table 6, above.

Such antibody fragments can be conjugated with effectors and used as provided herein.

### EXAMPLES

The following examples should not be construed as limiting the scope of this disclosure.

### Example 1 Engineering and Purification of Fab Constructs

Constructs were synthesized and subcloned into a pCEP mammalian expression vector (Invitrogen). The IgG1 Fab constructs were engineered to include the heavy chain C-terminal sequence DKTHTCAA (SEQ ID NO:44). The IgG2 Fab constructs (Fab 3 and Fab 4) were engineered to include the heavy chain C-terminal sequence ERKCAA (SEQ ID NO: 45). The IgG4 Fab constructs (Fab 9 and Fab 10) were engineered to have the heavy chain C-terminal sequence ESKYGCAA (SEQ ID NO:46) The Fab construct sequences are shown in Table 4, where engineered cysteines are in boldface and underlined, and substituted cysteines are in boldface and italics, and additionally substituted residues are shown as double underlined.

All Fab constructs were transiently expressed using the 293F system (Invitrogen®). Cells were grown to 600 mL using F17 media supplemented with 4 mM L-glutamine and 0.1% Pluronic® F-68 (BASF®) in 5% CO₂ to a density of 1.7 million cells/mL in a 2 L flask, and then transfected with 1 μg of DNA and 2.5 μg high molecular weight polyethyleneimine/mL of cells. After six days, the proteins were harvested by centrifuging the cells at 4000×g and filtered using a 0.22 μm filter.

The filtered supernatant was incubated with CaptureSelect™ IgG1-CH1 affinity matrix (Life Technologies) for one hour at room temperature with agitation. The slurry was filtered, poured into a column, and equilibrated with PBS. The bound protein was eluted with 100 mM glycine pH 3.0, neutralized with 1M Tris to a pH of 5.5, and filtered with a 0.2 μm filter.

Purified proteins were then analyzed using SDS-PAGE analysis. For non-reduced gels, 5 ug of purified protein, 5 μl of water, and 5 μl of NuPage® LDS Sample Buffer (Life Technologies) were mixed and incubated at 95° C. for 5 minutes. The samples were run on a 4-12% SDS-PAGE gel for 35 minutes at 200 mV. For reduced gels, the same protocol was followed, except that 2-Mercaptoethanol was added to the sample buffer to a final concentration of 1.2M.

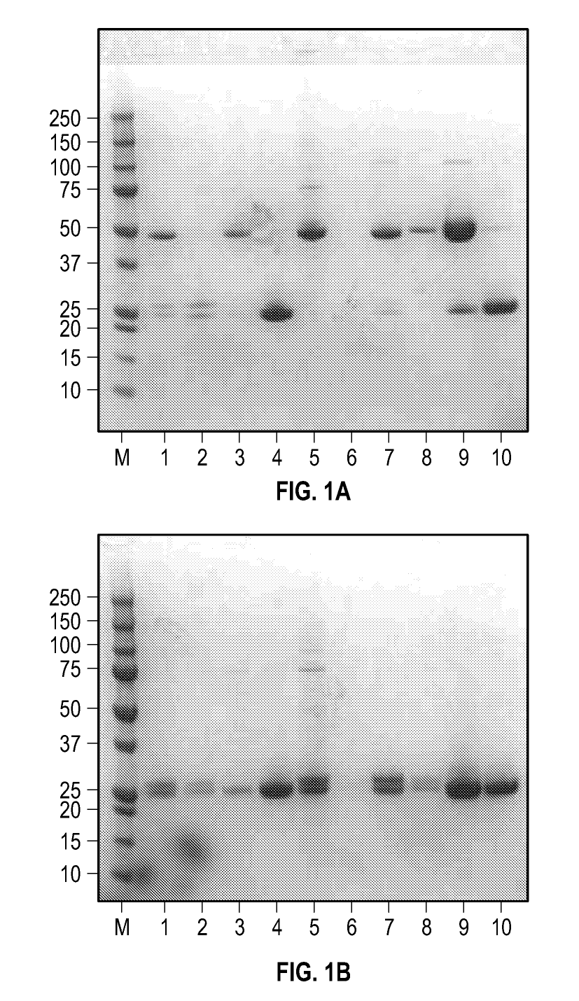

FIGS. 1-3 show the results of SDS-PAGE analysis of the purified Fabs. The description of the proteins run in each lane is in the figure legend. FIG. 1A shows the results of samples that were analyzed under non-reducing, non-denaturing conditions. Samples that had a disulfide bond migrated at approximately 50 kDa, with only some lower molecular weight bands being observed (corresponding to the V_{H}C_{H}1 (the slower migrating band of the doublet at approximately 25 kDa, e.g., lane 1); and V_{L}C_{L} chains (the faster migrating band of the doublet at approximately 22 kDa, e.g., lane 1). Thus for Fab 1, Fab 3, Fab 5, Fab 7, Fab 8 and Fab 9, where the native disulfide bonds were left intact or engineered, the bands co-migrated as intact Fabs. Fab 2, Fab 4, and Fab 10, which were without any paired cysteines and thus were not bonded to each other, migrated faster on the gel than Fab 1, Fab 3, Fab 5, Fab 7 and Fab 8. In FIG. 1B, where the samples were reduced and denatured, all samples migrated as doublets that corresponded to the Fab V_{H}C_{H}1 and V_{L}C_{L} chains. This observation demonstrated that cysteines can be engineered into Fab constructs that form disulfide bonds that maintain the bonds under non-denaturing, non-reducing conditions, but not in denaturing, reducing conditions.

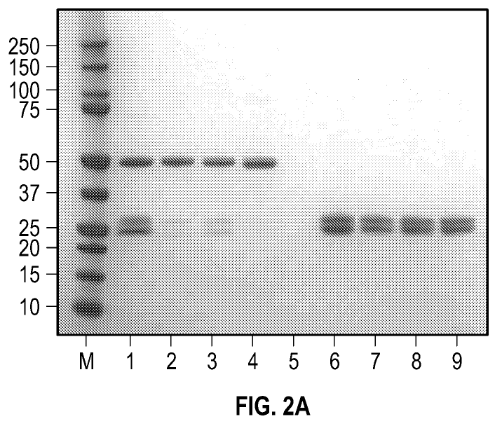

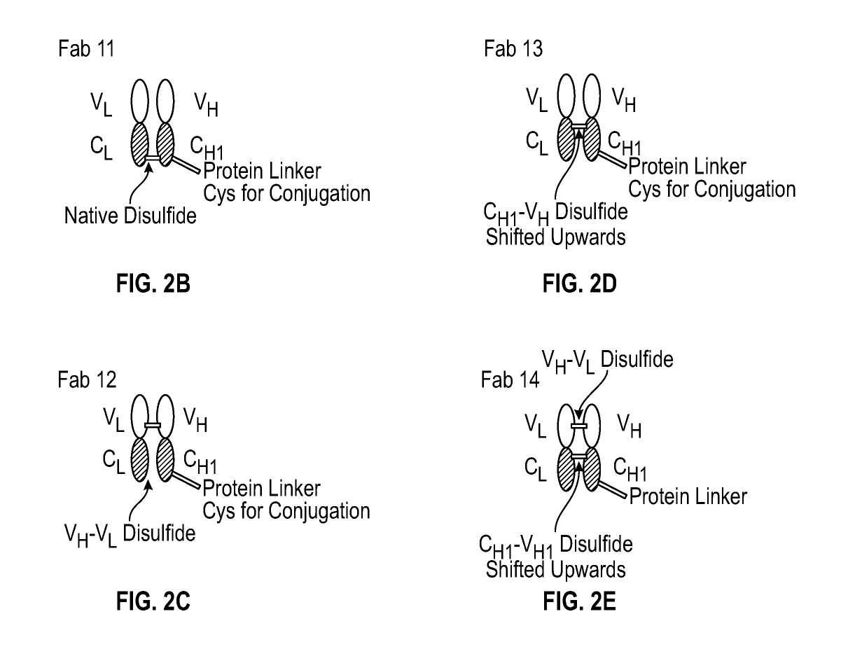

FIG. 2A shows the results of the SDS-PAGE analysis of Fabs 11-14. The description of the proteins run in each lane is in the figure legend. Fab 11, an unengineered Fab with the C-terminal disulfide bond is in lane 1. This lane contains both the Fab at 50 kDa, as well as lower molecular weight species. Lanes 2-4 show the non-reduced, non-denatured Fab 12, Fab 13, and Fab 14. In contrast to Fab 11, these lanes contain primarily the correct molecular weight species (50 kDa). Lanes 6-9 show Fabs 11-14 with the samples reduced and denatured, and all samples migrated as doublets that correspond to the Fab V_{H}C_{H}1 and V_{L}C_{L} chains. FIGS. 2B-2E show schematics of Fab 11, Fab 12, Fab 13, and Fab 14 constructs, respectively, illustrating the location of the disulfide bonds, such as in a wild-type Fab (Fab 11; FIG. 2B) and three engineered constructs having relocated disulfide bonds (Fab 12; FIG. 2C; Fab 13; FIG. 2D; Fab 14, FIG. 2E).

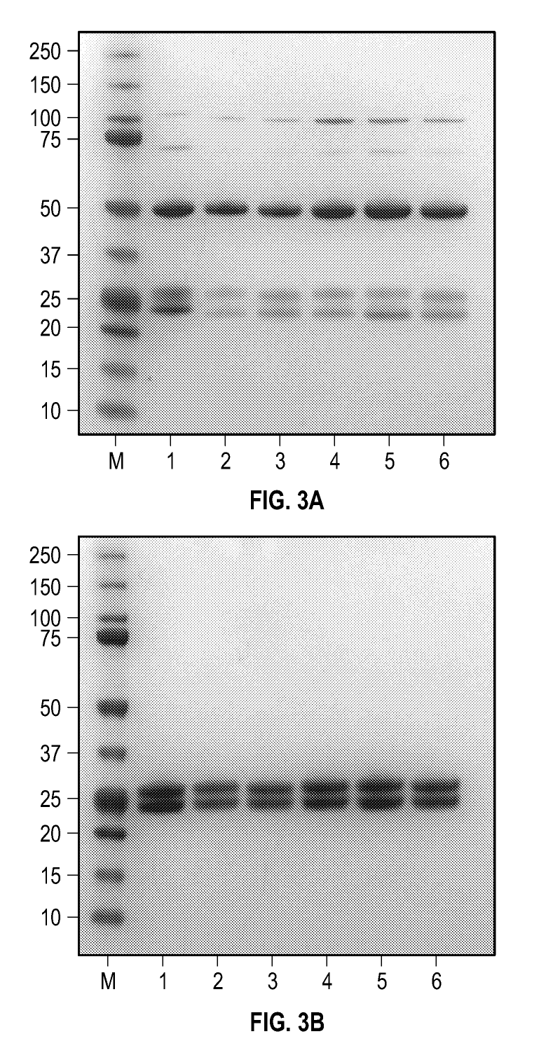

FIG. 3 shows the results of SDS-PAGE for Fab 11, and Fab 15-19. The description of the proteins run in each lane is in the figure legend. FIG. 3A shows the results of samples that were analyzed under non-reducing, non-denaturing conditions. Samples that had a disulfide bond migrated at approximately 50 kDa, with only some lower molecular weight bands being observed (corresponding to the V_{H}C_{H}1 (the slower migrating band of the doublet at approximately 25 kDa, e.g., lane 1); and V_{L}C_{L} chains (the faster migrating band of the doublet at approximately 22 kDa, e.g., lane 1). Lane 1 contains the protein with the native disulfide bond; the engineered Fabs (Lanes 2-6) show reduced lower molecular weight species. Lanes 6-9 show Fab 11, Fabs 15-19 with the samples reduced and denatured, and all samples migrated as doublets that correspond to the Fab V_{H}C_{H}1 and V_{L}C_{L} chains.

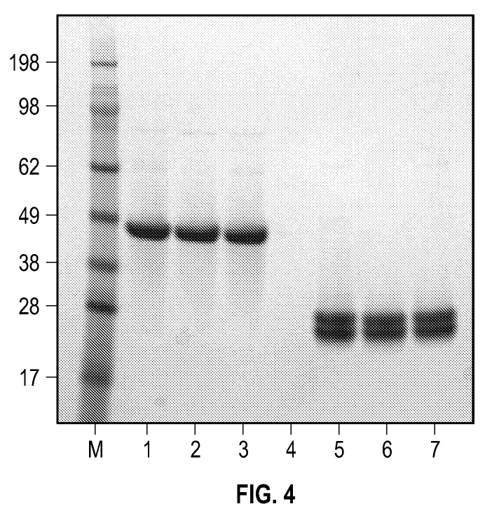

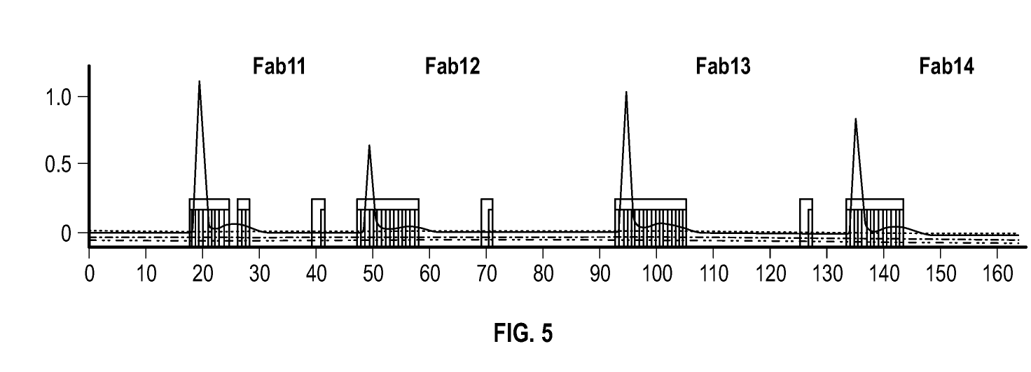

FIG. 4 shows the results of SDS-PAGE for Fab 20-22. Lanes 1-3 shows the results of samples that were analyzed under non-reducing, non-denaturing conditions. These engineered Fabs migrated to a molecular weight of approximately 49 kDa. Lanes 5-7 show Fabs 20-22 with the samples reduced and denatured, and all samples migrated as doublets that correspond to the Fab V_{H}C_{H}1 and V_{L}C_{L} chains.

### Example 2 Thermostability Analysis of Engineered Fabs

Purified Fabs were further analyzed to determine their melting temperatures. Melting temperatures were determined by differential scanning fluorescence. For Fabs 1-Fab 14, 10 μM of protein and 1× Sypro Orange (Life Technologies) in 1×PBS was mixed to a final volume of 25 μl and heated from 20° C. to 90° C. at a rate of 1° C./min using the IQ5 real time detection system (Bio-Rad). For Fab 15-Fab 24, 10 μM of protein and 1× of Protein Thermal Shift Buffer and Dye (Life Technologies) was mixed to a final volume of 20 μl and heated from 25° C. to 99° C. at a rate of 3° C./min using the Viia7 real time detection system (Life Technologies). The melting temperature reported is the temperature of the maximum value of the first derivative. The melting temperatures are reported in Table 8.

TABLE 8
Melting Temperatures of Fab constructs

| Construct | Tm |
|---|---|
| Fab 1 | 78.5 |
| Fab 2 | 72.5 |
| Fab 3 | 75.7 |
| Fab 4 | 70.7 |
| Fab 5 | 77.7 |
| Fab 6 | did not express |
| Fab 7 | 75.7 |
| Fab 8 | 65.7 |
| Fab 9 | 75.7 |
| Fab 10 | 70.9 |
| Fab 11 | 77.3 |
| Fab 12 | 76.1 |
| Fab 13 | 76.1 |
| Fab 14 | 77.5 |
| Fab 15 | 79.6 |
| Fab 16 | 80.7 |
| Fab 17 | 75.1 |
| Fab 18 | 73.8 |
| Fab 19 | 74.8 |
| Fab 20 | 70.3 |
| Fab 21 | 71.2 |
| Fab 22 | 70.7 |
| Fab 23 | did not express |
| Fab 24 | did not express |

### Example 3 Conjugation of Fabs with Mal-PEG-DSPE

To prepare purified Fabs 1 through 24 (sequences set forth in Table 7) for conjugation with mal-PEG-DSPE (1,2-distearoyl-sn-glycero-3-phosphoethanolamine-N-[maleimide(polyethylene glycol)]), Fabs in solution in 0.1 glycine-HCl or 10 mM citrate, pH adjusted to about 6.0 with Tris-base, were concentrated on a YM-10 diafiltration membrane (Amicon) to about 4-5 mg/ml of the protein. Reduction/activation of the C-terminal cysteine present in the heavy chain sequences of each Fab was performed by adding EDTA to 5 mM and cysteine hydrochloride, pH 5.7 (adjusted with 1 M trisodium citrate) to 15 mM, followed by incubation at 30° C. for 1 hour. The solution was passed through a SEPHADEX G-25 (PD-10) column to exchange the protein into conjugation buffer (5 mM citrate, 1 mM EDTA, 140 mM NaCl, pH 6.0). Aliquots of the resulting protein solution were diluted with conjugation buffer, typically 5-10-fold, to a volume of 0.9 ml, mixed with (a) 0.1 ml of 1 M HEPES-Na buffer pH 7.3, and (b) 0.01 ml of 20 mM, 5,5′-dithiobis(2-nitrobenzoic acid) (“Ellman's reagent”) in DMSO. 5-10 minutes after mixing, the absorbance of the solution was measured at 412 nm against a protein-free blank. Concentration of reactive thiol groups was calculated using the molar extinction value of 12,500 L/mol/cm and normalized to the molar concentration of the protein (A₂₈₀=1 molecular weight of kDa) determined by UV-spectrophotometry at 280 nm using molar extinction coefficient calculated from the protein's amino acid sequence (about 1.43).to give SH/protein ratio. The SH/protein ratios for the reduced Fabs are shown in Table 9. Ideally, the SH/protein ratio would be close to 1. As shown in the table, Fab 11, which contains wild-type disulfide bonds, has an SH/protein ratio of 1.64, suggesting the Fab is being over-reduced. Changing the disulfide pairing, such as Fab 13, Fab 15, Fab 16, Fab 17, Fab 18, Fab 19, Fab 20, Fab 21 and Fab 22, resulted in an improved SH/protein ratio, indicating the engineered Fabs are not being over-reduced.

Reduced Fabs were conjugated to mal-PEG-DSPE linker in the following way. First, mal-PEG-DSPE (PEG mol. weight 2000, NOF Corp., Japan) and methoxy-PEG-DSPE (PEG mol. weight 2000, Avanti Polar Lipids, USA) were co-dissolved in distilled water, acidified with citric acid to pH 5.7, at a concentration of 10 mg/ml each. The solution was briefly heated to 60° C. to effect the formation of mixed micelles containing thiol-reactive and nonreactive PEG-DSPE derivative. Then, the linker solution was added to 1 ml of the reduced protein solution in the conjugation buffer to achieve the mass ratio of the active (mal-PEG-DSPE) linker to the protein of 0.226 (molar ratio of about 3,45:1), and the conjugation mix was stirred at room temperature for about 4 hours. The reaction was stopped by quenching unreacted maleimide groups with 0.5 mM cysteine for 5-10 min, and, after analytical sampling, the mix was applied on a gravity-fed chromatography column with Ultrogel AcA 34 (Sigma Chemical Co, USA), bed volume 17 ml, equilibrated with the conjugate storage buffer (10% w/v sucrose, 10 mM citrate-Na, pH 6.5). The column was eluted with the same buffer, 0.5-ml fractions were collected, and the protein concentration was determined by spectrophotometry at 280 nm using the same extinction coefficients as for the unconjugated Fabs. Due to micellar character of the Fab-PEG-DSPE conjugate in aqueous solution (see, e.g., Nellis et al., 2005, Biotechnology Progress, v. 21, p. 221-232), the conjugate appeared in the fractions near the column void volume (first peak). These fractions were combined and passed through a 0.2-μm polyethersulfone syringe filter to give the purified conjugate. The second (smaller) protein peak, containing unconjugated protein, was detected and sampled for analysis. Table 9 presents the reactive thiol/protein ratios and Fab-PEG-DSPE conjugate yields across the engineered Fab variants, as well as for the “wild type” (native) Fab.

TABLE 9
Reduction and conjugation yield of Fabs

| | | Conjugate yield |
|---|---|---|
| Fab | SH/protein | (of reduced protein), % |
| 11 | 1.64 | 62.5 |
| 12 | 1.94 | 57.9 |
| 13 | 1.16 | 66.2 |
| 14 | 1.92 | 48.5 |
| 15 | 1.11 | 69.5 |
| 16 | 1.10 | 83.1 |
| 17 | 1.09 | 79.5 |
| 18 | 1.44 | 79.5 |
| 19 | 1.17 | 76.9 |
| 20 | 1.08 | 69.1 |
| 21 | 1.04 | 74.5 |
| 22 | 1.04 | 78.1 |

The Fabs and Fab conjugates were further assayed by non-reducing SDS-PAGE (NuPage® 1.0×12 Bis-Tris 4-15% gel; Life Technologies), SimplyBlue™ stain (Life Technologies), 2 μg/lane.

FIG. 6 shows SDS-PAGE of Fab 11, Fab 12, Fab 13, and Fab 14 as non-reduced and reduced Fabs prior to conjugation, the conjugation mix, the purified conjugation, and the unconjugated fraction. It was observed that purified Fab 13 gave a low proportion of dissociated chains as well as a low proportion of the multiple conjugated by-products (proteins with more than one linker attached) as can be seen in lane 15. Further, it was observed that Fab 12 produced a high proportion of chain dissociation products, as shown in lane 9. Fab 14 produced a low proportion of chain dissociation products, but a high proportion of multiple-conjugation products (the higher molecular weight species in lane 20). Fab 13 was selected for further engineering.

FIG. 7 shows SDS-PAGE of Fab 11, Fab 15 Fab 16, Fab 17, Fab 18, and Fab 19 as non-reduced protein, reduced protein, conjugation mix, and purified conjugates. Among these Fabs, Fab 16 (lane 12) and Fab 19 (lane 24) gave the lowest amount of dissociated chains; however, all were much better than the wild type (Fab 11, lane 4).

FIG. 8 shows SDS-PAGE of Fab 20, Fab 21, and Fab 22 as non-reduced protein, reduced protein, conjugation mix, and purified conjugate. Lane 4 (Fab 20), lane 8 (Fab 21), and lane 12 (Fab 22) show the purified conjugates. There is a single band in each of these lanes, demonstrating that these engineered Fabs produced conjugates, each with a single conjugated linker. This shows that these Fabs have high stability against chain dissociation during conjugation and good insertability into liposomes.

The Fab-PEG-DSPE conjugates were assayed for EphA2 binding strength using the FortéBIO® Octet® Red 96 system (Pall Corporation) to determine whether conjugation or engineering of the Fab affected binding activity. The results showed they did not. Anti-His5 sensors were first coated with his-tagged recombinant, human EphA (SEQ ID NO:47) at a concentration of 10 μg/ml protein in PBS. The sensors were then incubated in 4 μg/ml of Fab-PEG-DSPE conjugate in PBS. The slope of an association curve between 2-10 seconds was determined and compared across the variants and to the reference conjugate, Fab 11-PEG-PE, which is the anti-EphA2 antibody with wild-type disulfide pairing. The results are shown in Table 10. These results show that all of the Fabs, when conjugated via a Fab cysteine to mal-PEG-DPSE, retained at least 75% of their binding strength to EphA2 when compared to wild-type protein conjugate.

TABLE 10
EphA2 binding strength of Fab-PEG-DSPE conjugates
(relative to conjugates of wild type Fab (Fab 11)

| | Binding strength, |
|---|---|
| Fab | % of wild type conjugate |
| 12 | 108.3 |
| 13 | 96.9 |
| 14 | 105.0 |
| 15 | 86.0 |
| 16 | 85.0 |
| 17 | 89.7 |
| 18 | 75.5 |
| 19 | 96.8 |

### Example 4 Conjugation of Fabs to Liposomes

Liposomes of HSPC-Cholesterol-methoxyPEG(2000)DSPE (3:2:0.3 molar ratio) with an average size of 91 nm (PdI 0.06) were loaded with doxorubicin hydrochloride at the drug/liposome ratio of 0.13 g/mol phospholipid using ammonium sulfate gradient method (0.25 M ammonium sulfate) essentially as described by Martin (F. Martin, in: Injectable Dispersed Systems: Formulation, Processing, and Performance, ed. By D.Burgess, Informa Healthcare. New York, 2007, Ch. 14, p. 427-480). The lipids of the liposome were quantified by phosphate assay following acid digestion (W. R. Morrison, Anal. Biochem. Vol. 7, p. 218-224, 1964).

A solution of Fab-PEG-DSPE [PEG (2000)] conjugate in 10% sucrose-10 mM citrate buffer pH 6.5 was added to a suspension of liposomes in 10% sucrose, 10 mM histidine buffer pH 6.5, along with extra sucrose-citrate buffer to achieve concentrations of 0.16 mg/ml of the Fab and 8 mM of the liposome phospholipid (Fab/liposome ratio of 20 g protein/mol of phospholipid, or about 30 Fab molecules/liposome). The mixture was quickly heated to 60° C. and maintained at this temperature for 30 minutes with stirring. Then the mixture was chilled on ice, and the liposomes with membrane-inserted Fab-PEG-DSPE conjugates were separated from the non-inserted conjugate and extraliposomal drug by size-exclusion chromatography on a SEPHAROSE CL-4B column, eluted with 144 mM NaCl-5 mM HEPES buffer pH 6.5. The chromatography showed practically no leakage of the drug from the liposomes during the incubation, as judged by the absence of any visually detectable chromatographic band corresponding to free doxorubicin.

Aliquots of the liposomes containing known amounts of phospholipid were solubilized in SDS-PAGE running buffer and separated by SDS-PAGE on the NuPage® BT 4-12% gel (Life Technologies). The gels were stained with SimplyBlue™ Coomassie, and the bands were quantified by densitometry using concurrently run dilutions of bovine serum albumin (Pierce) as standards (FIG. 9 and Table 11). Any protein on the gel that was higher or lower molecular weight species than predicted for the conjugate was classified as non-product bands, and the percentage was calculated by comparing it to the density of the correct product band. As shown in Table 11, the wild-type Fab (Fab 11) had a high percentage of non-product bands: 45.2% for the conjugate and 24% for the conjugate-comprising liposomes. In contrast, the engineered Fabs exhibited a reduction of non-product bands. Using Fab 13, Fab 15, Fab17, Fab 18, Fab 19, Fab 20, Fab 21, or Fab 22 resulted in less than 10% non-product bands. The insertion efficiency was calculated as the percent of protein, per unit of phospholipid, that remained associated with the liposomes after purification by SEPHAROSE size-exclusion chromatography.

TABLE 11

| | Non-product bands | | |
|---|---|---|---|
| | (%) | | Insertion Efficiency |
| Fab | Conjugate | Liposomes | (%) |
| 11 | 45.2 | 24.0 | 63.4 |
| 12 | 27.5 | 16.6 | 69.5 |
| 13 | 6.4 | 2.2 | 84.9 |
| 14 | 23.4 | 10.1 | 42.4 |
| 15 | 8.2 | 11.0 | 92.8 |
| 16 | 15.0 | 14.3 | 93.7 |
| 17 | 9.4 | 8.8 | 84.2 |
| 18 | 7.6 | 9.5 | 93.3 |
| 19 | 8.6 | 8.1 | 87.4 |
| 20 | 5.3 | 4.5 | 95.5 |
| 21 | 5.5 | 4.5 | 99.5 |
| 22 | 5.8 | 3.4 | 90.1 |

The purified liposomes were assayed for EphA2 binding strength by FortéBIO® Octet® Red96 system (Pall) in PBS at 25 μM liposome phospholipid using anti-His5 sensors coated with recombinant human EphA2 with C-terminal hexahistidine (SEQ ID NO:47). The slope of the association curve from 3-20 sec was determined and compared to the slope observed for liposomes with inserted conjugate of the wild type Fab. The results are shown in FIG. 13. All liposomes with inserted Fab-PEG-DSPE conjugates having engineered Fabs showed greater than 80% EphA2 binding strength when compared to control matched liposomes with inserted Fab-PEG-DSPE conjugate of native Fab 11, indicating that the engineered Fabs were effectively incorporated into Fab-targeted drug-loaded liposomes.

TABLE 12
EphA2 binding strength of the doxorubicin liposomes with
inserted Fab-PEG-DSPE conjugates relative to the liposomes
with conjugate of the wild type Fab (Fab 11)

| | Fab-liposome binding |
|---|---|
| Fab | strength, % of control |
| 12 | 108.6 |
| 13 | 96.5 |
| 14 | 120.1 |
| 15 | 102.0 |
| 16 | 89.3 |
| 17 | 102.5 |
| 18 | 99.6 |
| 19 | 83.3 |

### Example 5 Engineered Forms of Additional Antibodies

This example demonstrates that the Fab constant regions described herein can function in the context of additional antibodies. Fab versions of anti-EGFR antibody P1X (U.S. Pat. No. 9,226,964), anti-EpCAM antibody MOC-31 (Roovers et al., 1998, Br. J. Cancer, 78:1407-16), and anti-HER2 antibody F5 (U.S. Pat. No. 9,226,966) having the same constant regions as wt Fab or Fab 7 were engineered and expressed essentially as described in Example 1. The engineered Fabs all bind with comparable affinity whether expressed as wt or with Fab 7 mutants.

The purified Fab proteins were analyzed using SDS-PAGE essentially as described in Example 1. All samples migrated at approximately 50 kDa, with only some lower molecular weight bands being observed. Under reducing conditions, all samples migrated as doublets that corresponded to the Fab VHCH1 and VLCL chains. This observation demonstrated that the Fab constructs formed disulfide bonds that were maintained under non-denaturing, non-reducing conditions, but not in denaturing, reducing conditions.

Thermal stability of the engineered Fabs was determined essentially as in Example 2 (Table 13). Where tested, the wt and Fab 7 versions of the antibodies had comparable melting temperatures.

TABLE 13
Melting temperatures of additional Fab constructs

| Construct | Tm (° C.) |
|---|---|
| P1X wt Fab | 80.57 |
| P1X Fab 7 | 80.58 |
| Moc31 Fab 7 | 71.36 |
| F5 Fab 7 | 86.18 |

### EQUIVALENTS

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure.

### INCORPORATION BY REFERENCE

The disclosure of each and every U.S. and foreign patent and pending patent application and publication referred to herein is specifically incorporated by reference herein in its entirety.

1. A Fab, said Fab comprising a heavy chain and a light chain;
said Fab characterized in that:
(a) there is not a cysteine at position 233 and at position 127 of the heavy chain and there is not a cysteine at position 214 of the light chain;
(b) the heavy chain and the light chain are linked together by one or two heavy-chain-light-chain disulfide bonds, each of the one or two bonds connecting a different pair of engineered cysteines located at positions selected from:
(i) position 44 of the heavy chain and position 100 of the light chain, and
(ii) position 174 of the heavy chain and position 176 of the light chain; and
(c) the heavy chain and light chain comprise:
(i) glutamic acid at heavy chain position 172 and aspartic acid at light chain position 162, or
(ii) phenylalanine at heavy chain position 172 and leucine at light chain position 162; wherein the numbering of the positions is according to the Kabat numbering system for IgG.

2. The Fab of claim 1, wherein the one or two heavy-chain-light-chain disulfide bonds is two bonds.

3. The Fab of claim 1, further comprising leucine at heavy chain position 44 and leucine at light chain position 100.

4. The Fab of claim 2, further comprising leucine at heavy chain position 44 and leucine at light chain position 100, and:
(i) glutamic acid at heavy chain position 172 and aspartic acid at light chain position 162; or
(ii) phenylalanine at heavy chain position 172 and leucine at light chain position 162 and valine at light chain position 174.

5. The Fab of claim 1, wherein the heavy chain and the light chain are selected from the group consisting of:
(a) a heavy chain having an amino acid sequence of SEQ ID NO:18 and a light chain having an amino acid sequence of SEQ ID NO:19;
(b) a heavy chain having an amino acid sequence of SEQ ID NO:20 and a light chain having an amino acid sequence of SEQ ID NO:21;
(c) a heavy chain having an amino acid sequence of SEQ ID NO:22 and a light chain having an amino acid sequence of SEQ ID NO:23;
(d) a heavy chain having an amino acid sequence of SEQ ID NO:24 and a light chain having an amino acid sequence of SEQ ID NO:25;
(e) a heavy chain having an amino acid sequence of SEQ ID NO:26 and a light chain having an amino acid sequence of SEQ ID NO:27;
(f) a heavy chain having an amino acid sequence of SEQ ID NO:28 and a light chain having an amino acid sequence of SEQ ID NO:29;
(g) a heavy chain having an amino acid sequence of SEQ ID NO:30 and a light chain having an amino acid sequence of SEQ ID NO:31; and
(h) a heavy chain having an amino acid sequence of SEQ ID NO:32 and a light chain having an amino acid sequence of SEQ ID NO:33;

6. The Fab of claim 1, further comprising at least one cysteine within 10 amino acid residues of the C-terminus of the heavy chain.

7. The Fab of claim 6, wherein the at least one cysteine is comprised within an amino acid sequence of SEQ ID NO:44 (DKTHTCAA) located at the C-terminus of the heavy chain.

8. The Fab of claim 1, wherein said Fab has a Tm of 70° C. or greater, as measured by a thermal shift assay using a differential scanning fluorimetry readout.

9. The Fab of claim 1, wherein said Fab has binding strength for its target antigen that is no less than 75% of that of a matched native, non-modified Fab.

10. (canceled)

11. The Fab of claim 6, wherein a moiety is attached to the cysteine.

12. The Fab of claim 11, wherein the moiety comprises a linker linking it to the cysteine.

13. The Fab of claim 11, wherein the linker is a cleavable linker.

14. (canceled)

15. A method of preparing the Fab of claim 11, wherein attachment of the moiety is accomplished by a maleimide thiol reaction between a 1,2-distearoyl-sn-glycero-3-phosphoethanolamine-N-[maleimide(polyethylene glycol)] linker and the cysteine.

16. The Fab of claim 1, wherein said Fab has increased stability, as measured by chain dissociation during moiety conjugation, when compared to a matched native Fab.

17. The Fab of claim 11, wherein the moiety comprises a lipidic nanoparticle.

18. (canceled)

19. A pharmaceutical composition comprising a Fab of claim 1, and one or more pharmaceutically acceptable excipients, diluents, or carriers.

20. A method of preparing a lipidic nanoparticle attached to a Fab by means of a linker molecule, the method comprising: attaching a Fab according to claim 1 to a linker molecule comprising a linear hydrophilic polymer chain having a first end and a second end, with, attached to the first end, a chemical group reacted with one or more functional groups on the Fab, and attached to the second end, a hydrophobic domain, optionally a lipid hydrophobic domain, and incubating the Fab-linker conjugate with the lipidic nanoparticle at a temperature of greater than 50°, 60°, or 70° C. for a time sufficient to permit the hydrophobic domain to become stably associated with the lipidic nanoparticle.

21. (canceled)

22. The method of claim 20, wherein the lipidic nanoparticle is a liposome comprising a cytotoxin.

23. (canceled)

24. The method of claim 20, wherein the linker is biodegradable.

25. The method of claim 20, wherein the insertion efficiency of a conjugate into a DSPC/Chol (3:2, mol:mol) 100 nm liposome is greater than 80% or greater than 90%.
